(19) United States
(12) Patent Application Publication
(10) Pub. No.: US 2025/0157674 A1
(43) Pub. Date: May 15, 2025

(54) RAPID SCALABLE RISK ASSESMENT FOR EMERGING VIRAL STRAINS

(71) Applicant: The University of Chicago, Chicago, IL (US)

(72) Inventor: Ishanu Chattopadhyay, Oaklawn, IL (US)

(21) Appl. No.: 18/821,279

(22) Filed: Aug. 30, 2024

Related U.S. Application Data
(60) Provisional application No. 63/535,650, filed on Aug. 31, 2023.

(51) Int. Cl. G16H 50/80; G16B 30/00

(52) U.S. Cl. CPC G16H 50/80; G16B 30/00

(57) ABSTRACT

Systems and methods are disclosed for predicting the rise of different strains of viruses. A method comprises reading a genetic sequence of a first strain of a virus; identifying a plurality of residue indices in the genetic sequence; for each of the plurality of indices, assigning a predictor, the predictor configured to predict a residue for its assigned index based upon a residue of at least one other index, the predictors thereby forming a network; and determining, based on the network of predictors, a probability of transition of the first strain to a second strain.

### RELATED APPLICATION(S)

This application claims the benefit of priority to U.S. Provisional Application No. 63/535,650, filed Aug. 31, 2023, which is incorporated herein by reference in its entirety.

### TECHNICAL FIELD

The disclosure is generally directed to a method of assessing viral strains such as influenza, and in particular, predicting emergent strains and identifying possible pandemic strains.

### SEQUENCE LISTING

This application contains a Sequence Listing which has been submitted electronically in XML format. The Sequence Listing XML is incorporated herein by reference. Said XML file, created on Jan. 13, 2025, is named UCT-01601_SL.xml and is 18,269 bytes in size.

### BACKGROUND OF THE DISCLOSURE

Animal influenza viruses emerging into humans have triggered devastating pandemics in the past. Yet, the ability to evaluate the pandemic potential of individual strains that do not yet circulate in humans, remains limited.

Accordingly, there is a need for systems and methods to computationally learn how new variants emerge, shaped by evolutionary constraints using only observed genomic sequences of key viral proteins.

### SUMMARY

According to certain aspects of the present disclosure, devices are disclosed for the ergonomic design of a surgical tool.

In one embodiment, a method comprises reading a genetic sequence of a first strain of a virus; identifying a plurality of residue indices in the genetic sequence; for each of the plurality of indices, assigning a predictor, the predictor configured to predict a residue for its assigned index based upon a residue of at least one other index, the predictors thereby forming a network; determining, based on the network of predictors, a probability of transition of the first strain to a second strain.

In some embodiments, the method further comprises determining, based on the probability of transition, that the second strain is above a dominant strain threshold; and providing a forecast of the dominant strain for a subsequent season.

In some embodiments, the dominant strain is a strain with a maximized probability of simultaneously arising from a set of currently circulating strains.

In some embodiments, the first strain is an animal strain of the virus and the second strain is a human strain of the virus.

In some embodiments, the method further comprises determining a pandemic potential of the first strain based upon the probability of transition of the first strain to a second strain.

In some embodiments, the method further comprises determining a divergence of each residue index of the plurality of residue indices; averaging the divergence over the plurality of residue indices in the genetic sequence; determining, based on the average of the divergence, a distance metric corresponding to the genetic sequence.

In some embodiments, the method further comprises based on the probability of transition, determining a risk score of the second strain, wherein the risk score indicates a risk of transmission to a human.

In some embodiments, the risk score is an average log likelihood of the probability of transition.

In some embodiments, the method further comprises reading a library of strains of the virus; determining a distance value between the second strain and each of the library of strains; and determining, from the distance values, a high-risk subset of the library.

In some embodiments, the virus is an influenza.

In some embodiments, the sequence is a genomic sequence.

In some embodiments, each of the residue indices corresponds to one or more surface proteins.

In some embodiments, each predictor comprises a conditional inference tree, a regression tree, and/or a decision tree.

In some embodiments, determining the probability of transition comprises: inferring a variation of a mutational probability of the sequence and potential residue replacements between the plurality of residue indices; and determining a probability of a spontaneous transition based on the inferred variation and the potential residue replacement.

In some embodiments, the probability of transition is a conditional distribution.

In some embodiments, the second strain comprises a human strain of a same subtype of the first strain, wherein the first strain is associated with a first season and the second strain is associated with a second season subsequent to the first season.

In some embodiments, the method further comprises searching a plurality of seasons prior to the first season for one or more historical strains of the same subtype.

In some embodiments, the method further comprises recomputing the probability of transition for each season.

In an alternative embodiment, a system comprises a datastore; a computing node comprising a computer readable storage medium having program instructions embodied therewith, the program instructions executable by a processor of the computing node to cause the processor to perform a method comprising: reading a genetic sequence of a first strain of a virus from the datastore; identifying a plurality of residue indices in the genetic sequence; for each of the plurality of indices, assigning a predictor, the predictor configured to predict a residue for its assigned index based upon a residue of at least one other index, the predictors thereby forming a network; determining, based on the network of predictors, a probability of transition of the first strain to a second strain.

In an alternative embodiment, computer program product for determining a probability of transition of a virus, the computer program product comprising a computer readable storage medium having program instructions embodied therewith, the program instructions executable by a processor to cause the processor to perform a method comprising: reading a genetic sequence of a first strain of a virus; identifying a plurality of residue indices in the genetic sequence; for each of the plurality of indices, assigning a predictor, the predictor configured to predict a residue for its assigned index based upon a residue of at least one other index, the predictors thereby forming a network; determining, based on the network of predictors, a probability of transition of the first strain to a second strain.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments.

FIG. 1A illustrates analysis of variations of genomes for identical subtypes of influenza A, according to techniques disclosed herein.

FIG. 1B illustrates a snapshot of decision trees, according to techniques disclosed herein. FIG. 1B discloses SEQ ID NOs 1-3 and 3, respectively, in order of appearance.

FIG. 1C illustrates a first application of forecasting dominant strain(s) for the next flu season, according to techniques disclosed herein.

FIG. 1D illustrates a second application of estimating the pandemic risk posed by individual animal strains that are still not known to circulate in humans, according to techniques disclosed herein.

FIGS. 2A-B illustrate the relative gains computed for different subtypes and hemispheres, according to techniques disclosed herein.

FIG. 3A illustrates a calculated approximate linear relationship between the average E-distance from human circulating strains and the published IRAT emergence scores calculated by the CDC, according to techniques disclosed herein.

FIG. 3B illustrates an estimation of the IRAT emergence score via fitting a GLM model to the E-distances estimated from the Emergenet, according to techniques disclosed herein.

FIG. 3C illustrates an estimate of IRAT impact scores via fitting a separate GLM model to the E-distances, according to techniques disclosed herein.

FIG. 3D illustrates a global prediction of IRAT scored for all influenza A sequences collected since 2020, according to techniques disclosed herein.

FIG. 4 illustrates a standard phylogeny constructed with edit distances, with all Influenza A strains collected between January 2020 and September 2022, with estimated IRAT emergence risk >6.0, and top strains which have IRAT scores, according to techniques disclosed herein.

FIG. 5A compares the sequences of Emergenet and the WHO recommendation, according to techniques disclosed herein. FIG. 5A discloses SEQ ID NOs 4-6, respectively, in order of appearance.

FIG. 5B illustrates the localization of the deviations in the molecular structure of HA, according to techniques disclosed herein.

FIG. 6 illustrates an HA sequence comparison with 2020-2021 dominant human strains (A/Baltimore/JH/001/2021, A/Myanmar/I026/2021, A/Darwin/12/2021) with Emergenet estimated top H3N2 risky strain EPI1818137 (2020-2022 April) showing differences in and out of the RBD, according to techniques disclosed herein. FIG. 6 discloses SEQ ID NOs 7-10, respectively, in order of appearance.

FIG. 7 illustrates HA sequence comparison with dominant human strains (A/Togo/0172/2021, A/Bangladesh/9004/2021, A/Wisconsin/04/2021) with Emergenet estimated top H1N1 risky strain EPI1818121 (2020-2022 April) showing differences both in and out of the RBD, according to techniques disclosed herein. FIG. 7 discloses SEQ ID NOs 11-14, respectively, in order of appearance.

FIGS. 8A-E illustrate E-distance validation in-silico using Influenza A sequences from NCBI databases, according to techniques disclosed herein.

FIG. 9 illustrates a number of mutations from the seasonal dominant strain over the years.

FIG. 10 is a series of histograms showing the sampling effects on E-distance.

FIG. 11 is an exemplary computing node.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

Influenza viruses constantly evolve, sufficiently altering surface protein structures to evade the prevailing host immunity, and cause the recurring seasonal epidemic. These periodic infection peaks claim a quarter to half a million lives globally, and the current response hinges on annually inoculating the human population with a reformulated vaccine. Among numerous factors that hinder optimal design of the flu shot, failing to correctly predict the future dominant strain dramatically reduces vaccine effectiveness. Despite recent advances, such predictions remain imperfect. In addition to the seasonal epidemic, influenza strains spilling over into humans from animal reservoirs have triggered pandemics at least four times (1918 Spanish flu/H1N1, 1957 Asian flu/H2N2, 1968 Hong Kong flu/H3N2, 2009 swine flu/H1N1) in the past 100 years. With the memory of the sudden SARS-CoV-2 emergence fresh in minds, a looming question is whether such events can be preempted and mitigated in the future. Influenza A, partly on account of its segmented genome and its wide prevalence in common animal hosts, can easily incorporate genes from multiple strains and (re)emerge as novel human pathogens, thus harboring a high pandemic potential.

One possible approach to mitigating such risk is to identify animal strains that do not yet circulate in humans, but are likely to spill-over and quickly achieve human-to-human (HH) transmission capability. While global surveillance efforts collect wild specimens from diverse hosts and geo-locations annually, the ability to objectively, reliably and scalably risk-rank individual strains remains limited, despite some recent progress.

The Center for Disease Control's (CDC) current solution to this problem is the Influenza Risk Assessment Tool (IRAT). Subject matter experts (SME) score strains based on the number of human infections, infection and transmission in laboratory animals, receptor binding characteristics, population immunity, genomic analysis, antigenic relatedness, global prevalence, pathogenesis, and treatment options, which are averaged to obtain two scores (between 1 and 10) that estimate 1) the emergence risk and 2) the potential public health impact on sustained transmission. IRAT scores are potentially subjective, and depend on multiple experimental assays, possibly taking weeks to compile for a single strain. This results in a scalability bottleneck, particularly with thousands of strains being sequenced annually.

Here, a pattern recognition algorithm to automatically parse out emergent evolutionary constraints operating on Influenza A viruses in the wild is introduced, to provide a less-heuristic, theory-backed scalable solution to emergence prediction. This approach is centered around numerically estimating the probability Pr(x→y) of a strain x spontaneously giving rise to y. It is shown that this capability is key to preempting strains which are expected to be in future circulation, and 1) reliably forecast dominant strains of seasonal epidemics, and 2) approximate IRAT scores of non-human strains without experimental assays or SME scoring.

In some embodiments, 405,778 Haemagglutinnin (HA) and Neuraminidase (NA) sequences from public databases are analyzed. The likelihood of specific future mutations is estimated, as well as the numerical odds of specific descendants arising via natural processes. After validating a model to forecast the dominant strain(s) for seasonal flu, with Emergenet-based forecasts significantly outperforming WHO recommendations consistently over the past two decades for H1N1/H3N2 subtypes, individually in the Northern/Southern hemispheres (average match-improvement 52.83% over two decades, 153.32% over the last decade, and 159.70% over the pre-COVID-19 five year period for H1N1 HA), the pandemic potential of animal strains not yet known to transmit in humans is assessed. While the state-of-the-art Influenza Risk Assessment Tool (IRAT) from the CDC to assess such risk includes time-consuming experimental assays, embodiments of the present disclosure include calculations taking ˜6 sec/strain, strongly correlating with published IRAT scores (correlation: 0:707, p-value: 0:00024). This six-orders-of-magnitude speedup is necessary to exploit current surveillance capacity, and analyze thousands of strains collected annually. Considering 6,066 wild Influenza A animal viruses sequenced post-2020, risky strains of diverse subtypes, hosts and geo-locations are identified, with six having estimated emergence scores >6:5. Such scalable risk-ranking can enable preemptive pandemic mitigation, including the targeted inoculation of animal hosts before the first human infection, and outline new public health measures that are potentially effective notwithstanding possible vaccine hesitancy in humans.

### Emergenet Inference

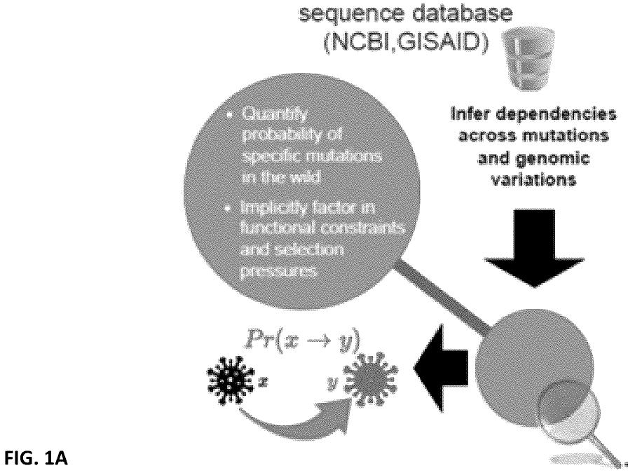

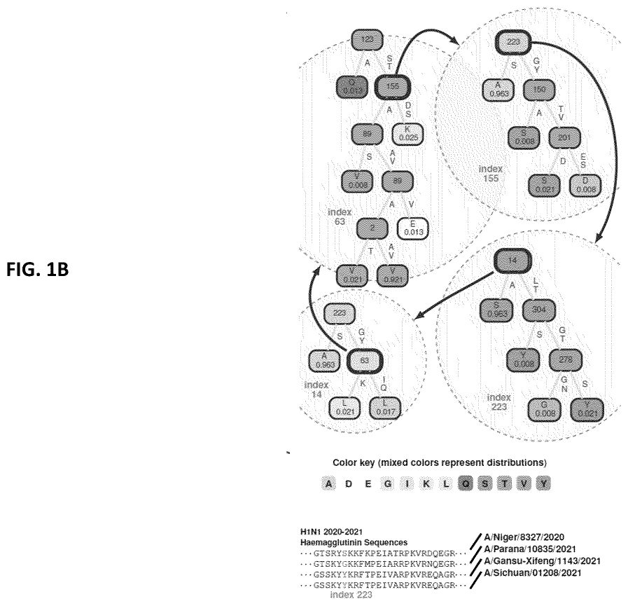

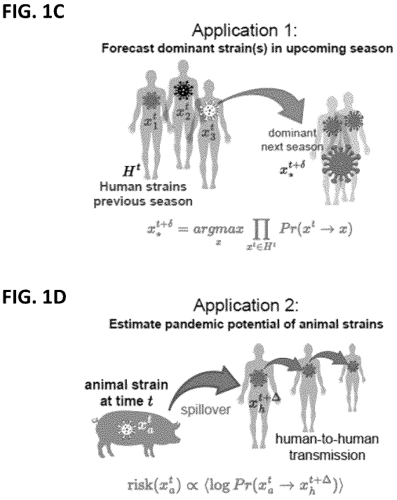

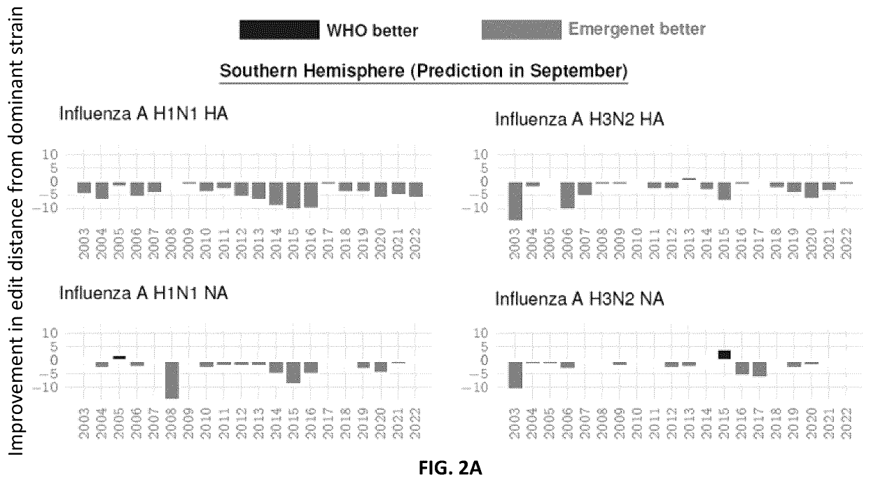

To uncover relevant evolutionary constraints, variations (point substitutions and indels) of the residue sequences of key proteins implicated in cellular entry and exit, were analyzed, namely HA and NA respectively. By representing these constraints within a predictive framework—the Emergenet (Enet)—the odds of a specific mutation to arise in the future were estimated, and consequently the probability of a specific strain spontaneously evolving into another (FIG. 1A). As shown in FIG. 1A, variations of genomes for identical subtypes of influenza A are analyzed to infer a recursive forest of conditional inference trees—the Emergenet—that maximally captures the emergent dependencies between an a priori unspecified number of mutations. With these inferred dependencies, it is possible to estimate the numerical odds of specific mutations, and by extension, the numerical value of the probability of one strain giving rise to another in the wild, under complex selection pressures from the background.

Such explicit calculations are difficult without first inferring the variation of mutational probabilities and the potential residue replacements from one positional index to the next along the protein sequence. The many well-known classical DNA substitution models or standard phylogeny inference tools which assume a constant species-wise mutational characteristics, are not applicable here. Similarly, newer algorithms such as FluLeap which identifies host tropism from sequence data, or estimation of species-level risk do not allow for strain-specific assessment.

The dependencies uncovered are shaped by a functional necessity of conserving/augmenting fitness. Strains must be sufficiently common to be recorded, implying that the sequences from public databases that are trained with have high replicative fitness. Lacking kinetic proofreading, Influenza A integrates faulty nucleotides at a relatively high rate (10⁻³-10⁻⁴) during replication. However, few variations are actually viable, leading to emergent dependencies between such mutations. Furthermore, these fitness constraints are not time-invariant. The background strain distribution, and selection pressure from the evolution of cytotoxic T lymphocyte epitopes in humans can change quickly. With a sufficient number of unique samples to train on for each flu season, the Emergenet (recomputed for each time-period) is expected to automatically factor in the evolving host immunity, and the current background environment.

Structurally, an Emergenet comprises an interdependent collection of local predictors, each aiming to predict the residue at a particular index using as features the residues at other indices (FIG. 1B). FIG. 1B illustrates a snapshot of a collection of decision trees from the Emergenet inferred for H1N1 HA sequence collected in 2020-2021, which reveals a cyclic dependency. In general, each internal node of a component tree can be “expanded” into its own tree, underscoring the recursive structure of the Emergenet.

An Emergenet comprises almost as many such position-specific predictors as the length of the sequence. These individual predictors are implemented as conditional inference trees11, in which nodal splits have a minimum pre-specified significance in differentiating the child nodes. Thus, each predictor yields an estimated conditional residue distribution at each index. The set of residues acting as features in each predictor are automatically identified, e.g., in the fragment of the H1N1 HA Emergenet (2020-2021, FIG. 1B), the predictor for residue 63 is dependent on residue 155, and the predictor for 155 is dependent on 223, the predictor for 223 is dependent on 14, and the residue at 14 is again dependent on 63, revealing a cyclic dependency. The complete Emergenet harbors a vast number of such relationships, wherein each internal node of a tree may be “expanded” to its own tree. Owing to this recursive expansion, a complete Emergenet substantially captures the complexity of the rules guiding evolutionary change as evidenced by the out-of-sample validation.

Structurally, an Emergenet comprises an interdependent collection of local predictors, each aiming to predict the residue at a particular index using as features the residues at other indices (FIG. 1B). Thus, an Emergenet comprises almost as many such position-specific predictors as the length of the sequence. These individual predictors are implemented as conditional inference trees, in which nodal splits have a minimum pre-specified significance in differentiating the child nodes. Thus, each predictor yields an estimated conditional residue distribution at each index. The set of residues acting as features in each predictor are automatically identified, e.g., in the fragment of the H1N1 HA Emergenet (2020-2021, FIG. 1B), the predictor for residue 63 is dependent on residue 155, and the predictor for 155 is dependent on 223, the predictor for 223 is dependent on 14, and the residue at 14 is again dependent on 63, revealing a cyclic dependency. The complete Emergenet harbors a vast number of such relationships, wherein each internal node of a tree may be “expanded” to its own tree. Owing to this recursive expansion, a complete Emergenet substantially captures the complexity of the rules guiding evolutionary change as evidenced by the out-of-sample validation.

In a first application of predicting future dominant strains, the H1N1 and H3N2 HA and NA sequences from Influenza A strains in the public NCBI and GISAID databases recorded between 2000-2022 (393; 189 in total, Supplementary Table 1). Emergenets are separately constructed for H1N1 and H3N2 subtypes, and for each flu season using HA sequences, yielding 84 models in total for predicting seasonal dominance. Using only sequence data is advantageous since deeper antigenic characterization tends to be substantially low-throughput compared to genome sequencing. However, deep mutational scanning (DMS) assays have been shown to improve seasonal prediction 6. Despite limiting to only genotypic information (and subtypes), the approach distills emergent fitness-preserving constraints that outperform reported DMS-augmented strategies.

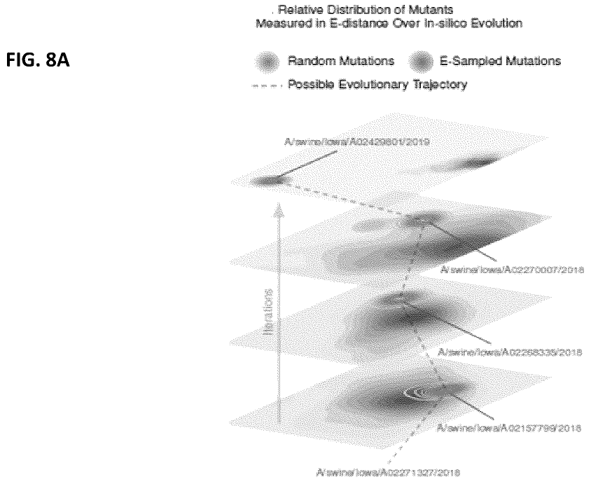

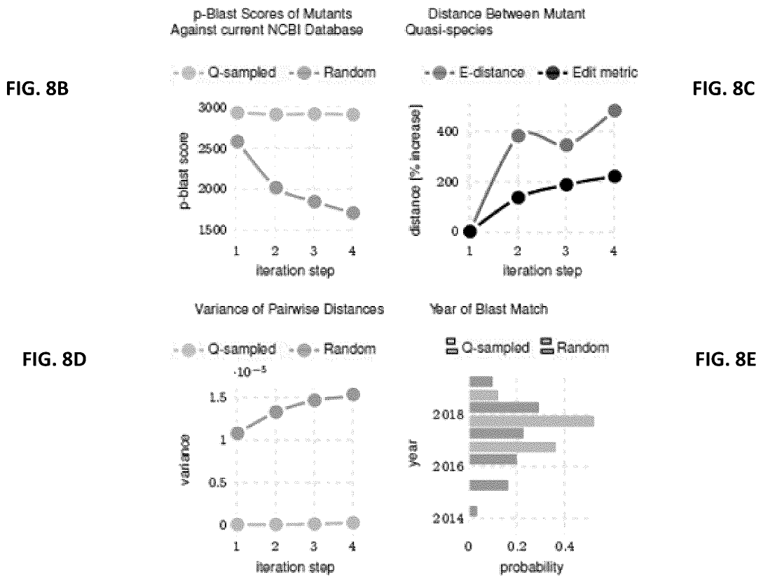

Inference of the Emergenet predictors is the first step, which then induces an intrinsic distance metric between strains. The E-distance (i.e., Emergenet distance) (Eq. (6) in Methods) is defined as the square-root of the Jensen-Shannon (JS) divergence of the conditional residue distributions, averaged over the sequence. Unlike the classical approach of measuring the number of edits between sequences, the E-distance is informed by the Emergenet-inferred dependencies, and adapts to the specific subtype, allele frequencies, and environmental variations. Central to the approach is the theoretical result (Theorem 1 in Methods) that the E-distance approximates the log-likelihood of spontaneous change, i.e., log Pr(x→y). With E-distance, the notion of “loci” is less important, as the distances between entire sequences are computed. This is a more general approach, since the use of loci ignores epistatic effects and cross-talk between non-collocated mutations. However, a SHAP analysis has been added to identify which loci are contributing most to the distance calculation, and it emerges that the RBD sites are indeed driving the distance differences between strains in the context of seasonal strain calculations. Note that despite general correlation between E-distance and edit-distance, the E-distance between fixed strains can change if only the background environment changes (Supplementary Table 2, 3). Thus, a new model using current data is learned, and only predicted for the near future. In in-silico experiments, it was found that while random mutations to genomic sequences produce rapidly diverging sets, Emergenet-constrained replacements produce sequences that are verifiably meaningful (In-silico Corroboration of Emergenet's Capability To Capture Biologically Meaningful Structure, Methods and FIG. 8).

It is true that a priori, the space of jumps from one sequence x to another sequence y is vast, and learning the exact model of jump from one strain to another is infeasible from the available data. However, the set of rules or constraints that best describe the data allows for the dramatic reduction of the set of future possibilities in a probabilistic sense. More precisely, each positional index in the HA primary structure is viewed individually as a target variable, and using all other indices as features to obtain a classification model, resulting in ˜566 conditional inference trees, as described above. Using conditional inference trees for the classification models implies it is guaranteed that only statistically significant splits are allowed in the inferred decision tree. When no such splits are found, no classification model is returned for that index. Finally, this forest of trees collectively comprises one Emergenet model, which induces the E-distance metric proven (Theorem 1 in Methods) to scale with the log-likelihood of the transition or jump probability. Determining the numerical odds of a spontaneous jump Pr(x→y) (FIG. 1C) allows the framing of the problem of forecasting dominant strain(s), and that of estimating the pandemic potential of an animal strain as mathematical propositions (albeit with some simplifying assumptions), with approximate solutions (FIG. 1C-D, Eq. (13)-(14) in Methods), which is discussed further in the upcoming two sections.

FIG. 1C illustrates a first application of forecasting dominant strain(s) for the next flu season, using only sequences collected up to six months prior and the inferred Emergenet, using data from the past year. FIG. 1D illustrates a second application of estimating the pandemic risk posed by individual animal strains that are still not known to circulate in humans (see Eq. (14) in Methods).

### Predicting Future Dominant Strains

A dominant strain for an upcoming season may be identified as one which maximizes the joint probability of simultaneously arising from each (or most) of the currently circulating strains (FIG. 1C). This does not deterministically specify the dominant strain, but a strain satisfying this criterion has high odds of acquiring dominance. And, a pandemic risk score of a novel strain may be estimated by the probability of the novel strain giving rise to a well-adapted human strain. In the context of forecasting future dominant strain(s), a search criteria is derived (further described in Predicting Future Dominant Seasonal Strains, Methods) from the above proposition, to identify historical strain(s) that are expected to be close to the next dominant strain(s):

$${x}_{*}^{t + δ} = \begin{matrix}arg min \\ y ∈ {U}_{τ \leq t} {H}^{τ}\end{matrix} ( {\sum }_{x ∈ {H}^{t}} {θ}^{[ t ]} ( x , y ) - \vert {H}^{t} \vert A ln {w}_{y} ) \qquad ( 1 )$$

where x_{*}^{t+δ} is a predicted dominant strain at time t+δ, H^{t} is the set of currently circulating human strains at time t observed over the past year, θ^{[t]} is the E-distance informed by the inferred Emergenet using sequences in H^{t}, w_{y} is the estimated probability of strain y being generated by the Emergenet, and A is a constant dependent on the sequence length and significance threshold used. The first term gets the solution close to the centroid of the current strain distribution (in the E-distance metric, and not the standard edit distance), and the second term relates to how common the genomic patterns are amongst recent human strains.

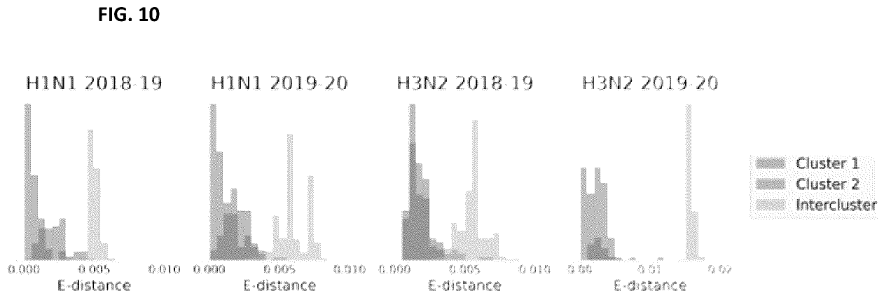

Prediction of the future dominant strain as a close match to a historical strain allows out-of-sample validation against past World Health Organization (WHO) recommendations for the flu shot, which is reformulated February and September of each year for the northern and southern hemispheres, respectively, based on a cocktail of historical strains determined via global surveillance. For each year of the past two decades, a separate Emergenet is constructed for the southern and northern flu seasons using HA strains available from the previous season (up to February for the north, and September for the south). For seasons with >3000 strains available, 3000 strains were randomly sampled to provide an accurate representation of the strain population. As shown in Supplementary Table 8 and FIG. 10, sampling has little effect on the results, given a large enough sample size. Using the Emergenet, the E-distance matrix was computed between HA sequences in the current population, H^{t}. H^{t} was then clustered using the E-distance matrix and the MeanShift clustering algorithm, such that there are n disjoint clusters H₁^{t}, H₂^{t}, . . . . Hₙ^{t}, with ∪nᵢ₌₁ⁿ, Hᵢ^{t}=H^{t}, where n is variable and determined by MeanShift based on the strain landscape. Thus, multiple predictions per season can be made by replacing H^{t} in Eq. (1) with H^{t} for i=1, 2, . . . n. In some embodiments, two recommendations are made per season, corresponding to the predictions from the two largest clusters.

FIG. 10 is a series of histograms showing the sampling effects on E-distance. One hundred Emergenet models were trained on the HA segment with different random samples of 3000 strains. A pair of random strains were sampled from each of the two largest clusters and compute their E-distance under each Emergenet model. The yellow represents the inter-cluster distance between the strains in the two clusters. There is little to no overlap between the same cluster E-distances and the inter-cluster distances. Note that the 100 E-distances between the random strains are upscaled match the number of inter-cluster E-distances.

To validate and compare recommendations, a representative sample of strains is needed in the next-year population. For each season, the MeanShift algorithm is again used to cluster the strain population using an edit distance matrix for both HA and NA segments separately. Each cluster Pᵢ^{t} is represented by a dominant strain \hat{x}^{dom-t}, defined as the strain closest to the centroid in the edit distance metric (L(x,y)) (Eq. (2)). The error is computed as the weighted average minimum edit distance between the two recommendations and the dominant strains, where the weight of the dominant strain \hat{x}^{dom-t} is the proportion of its cluster to the population, i.e., wᵢ^{t}=|Pᵢ^{t}|/|H^{t}|.

$${\hat{x}}^{dom - t} \begin{matrix}arg min \\ x ∈ {P}_{i}^{t}\end{matrix} {\sum }_{y ∈ {P}_{i}^{t}} L ( x , y ) \qquad ( 2 )$$

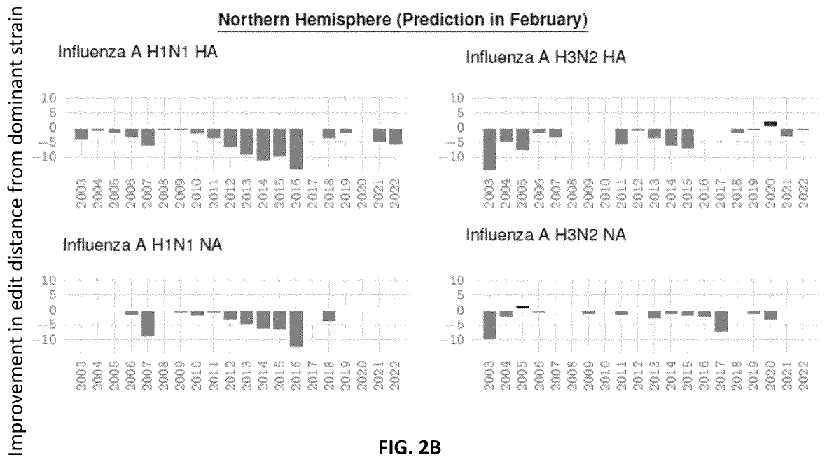

The Emergenet-informed forecasts outperform WHO/CDC recommended flu vaccine compositions consistently over the past two decades, for both H1N1 and H3N2 subtypes, individually in the northern and the southern hemispheres (which have distinct recommendations). For H1N1 HA, the Emergenet recommendation outperforms WHO by 52.83% on average over the last two decades, by 153.32% on average in the last decade, and by 159.70% in the period 2015-2019 (5 years pre-COVID-19). The gains for H1N1 NA over the same time periods are 34.48%, 86.46%, and 97.34% respectively. For H3N2 HA, the Emergenet recommendation outperforms WHO by 70.08% on average over the last two decades, by 45.86% on average in the last decade, and by 53.06% in the period 2015-2019. The gains for H3N2 NA over the same time periods are 52.13%, 55.44%, and 55.88%, respectively (Extended Data Table 2). Detailed predictions, along with historical strains closes to the observed dominant one are tabulated in Extended Data Tables 3 through 6. Visually, FIGS. 2A-B illustrate the relative gains computed for different subtypes and hemispheres.

FIGS. 2A-B shows the seasonal predictions for Influenza A. The relative out-performance of Emergenet predictions against WHO recommendations for H1N1 and H3N2 sub-types for the HA and NA coding sequences is evident over the both hemispheres. Predictions are done based on HA sequence data, and the corresponding NA sequences of the predicted strains are used for NA comparison here. The negative bars (red) indicate the reduced average edit distance between the predicted sequence and the representative dominant strains that year. Note that the recommendations for the north are given in February, while that for the south are given in September, keeping in mind that the flu season in the south begins a few months earlier (e.g. for the 2022-2023 flu season, northern data in the table is labelled ‘2022’).

The performance of the recommendation was also analyzed against two random strains selected from the last year's population. The two random strains from each season are random “predictions,” choosing from strains circulating in the past one year leading up to vaccine selection, and computing the errors using the same weighted average minimum edit distance method as used for the recommendations. This is repeated 20 times for each season and the upper bound of the 95% confidence interval is taken to account for the large variance in error as the worst case. If E is the set of 20 errors, this is

$$\overline{E} + 1.96 \times \frac{{s}_{E}}{20} .$$

For H1N1 HA, the Emergenet recommendation outperforms the random recommendation by 34.67% on average over the last two decades and by 68.07% on average in the last decade. The gains for H1N1 NA over the same time periods are 23.23% and 29.84%, respectively. For H3N2 HA, the Emergenet recommendation outperforms WHO by 66.82% on average over the last two decades and by 67.95% on average in the last decade. The gains for H3N2 NA over the same time periods are 36.92% and 34.05%, respectively (Extended Data Table 7).

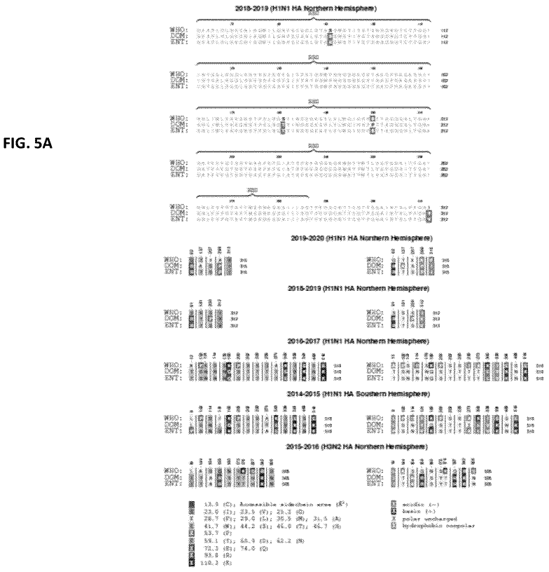

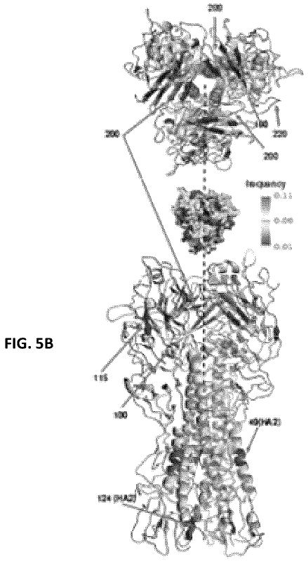

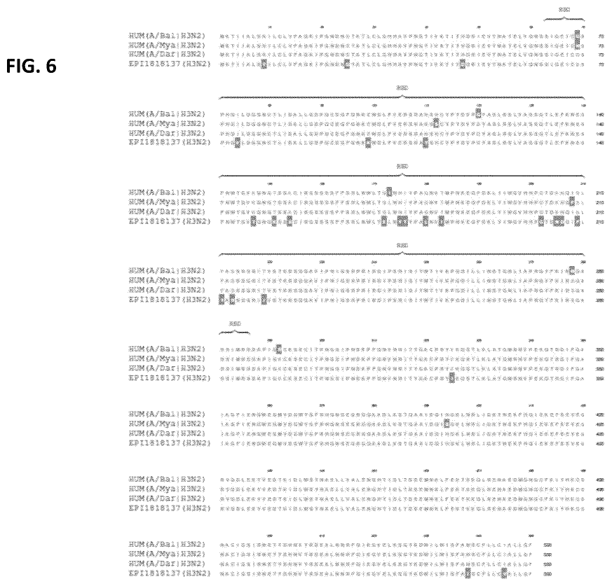

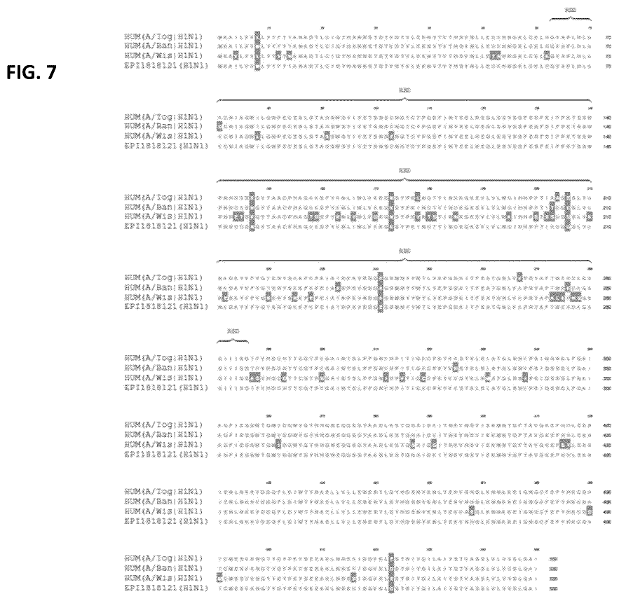

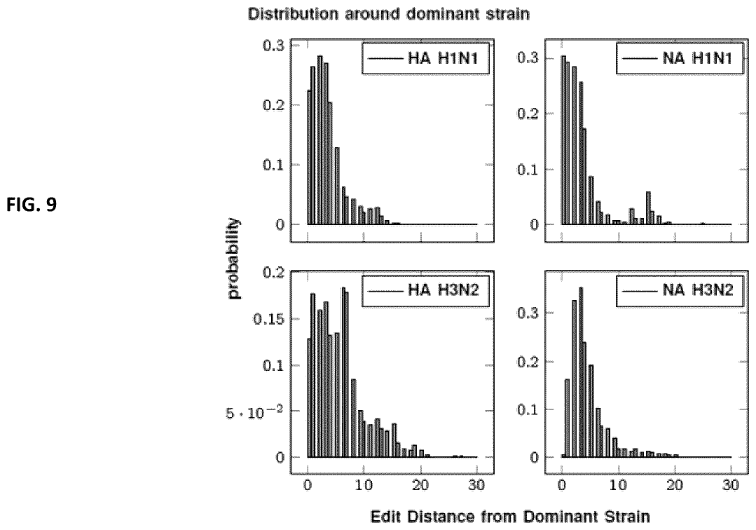

Comparing the Emergenet inferred strain (ENT) against the one recommended by the WHO, it is found that the residues that only the Emergenet recommendation matches correctly with dominant strain (DOM), while the WHO recommendation fails, are largely localized within the RBD, with >57% occurring within the RBD on average (FIGS. 5A and 7) when the WHO strain deviates from the ENT/DOM matched residue, the “correct” residue is often replaced in the WHO recommendation with one that has very different side chain, hydropathy and/or chemical properties (FIG. 5A), suggesting deviations in recognition characteristics. Combined with the fact that circulating strains are almost always within a few edits of the DOM (FIG. 9), these observations suggest that hosts vaccinated with the ENT recommendation can have season-specific antibodies that recognize a larger cross-section of the circulating strains.

FIG. 5B illustrates, by comparing the type, side chain area, and the accessible side chain area, that DOM and ENT are often close in important chemical properties, while WHO deviations are not (FIG. 5A). FIG. 5B shows the localization of the deviations in the molecular structure of HA, where it is noted that the changes are most frequent in the HA1 sub-unit (the globular head), and around residues and structures that have been commonly implicated in receptor binding interactions, e.g., the ˜200 loop, the ˜220 loop, and the ˜180 helix.

FIG. 6 illustrates an HA sequence comparison with 2020-2021 dominant human strains (A/Baltimore/JH/001/2021, A/Myanmar/I026/2021, A/Darwin/12/2021) with Emergenet estimated top H3N2 risky strain EPI1818137 (2020-2022 April) showing differences in and out of the RBD.

FIG. 7 illustrates HA sequence comparison with dominant human strains (A/Togo/0172/2021, A/Bangladesh/9004/2021, A/Wisconsin/04/2021) with Emergenet estimated top H1N1 risky strain EPI1818121 (2020-2022 April) showing differences both in and out of the RBD, according to techniques disclosed herein.

FIG. 9 illustrates the number of mutations from the seasonal dominant strain over the years. The quasispecies hat circulates each season for each sub-type is tightly distributed around the dominant strain on average.

### Estimating Pandemic Risk of Non-Human Strains

Some embodiments, however, have the ability to estimate the pandemic potential of novel animal strains, via a time-varying E-risk score ρₜ(x) for a strain x not yet found to circulate in human hosts. It is shown that (Measure of Pandemic Potential, Methods):

$${ρ}_{t} ( x ) {=}^{Δ} - \frac{1}{\vert {H}^{t} \vert} {\sum }_{y ∈ {H}^{t}} {θ}^{[ t ]} ( x , y ) \qquad ( 3 )$$

scales as the average log-likelihood of Pr(x→y) where y is any human strain of a similar subtype to x, and θ^{[t]} is the E-distance informed by the Emergenet computed from recent human strains H^{t} at time t of the same subtype as x, observed over the past year. As before, the Emergenet inference makes it possible to estimate ρₜ(x) explicitly.

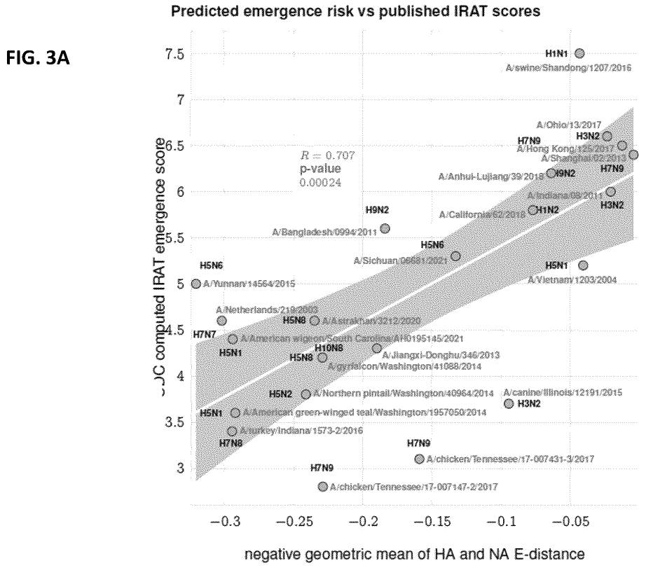

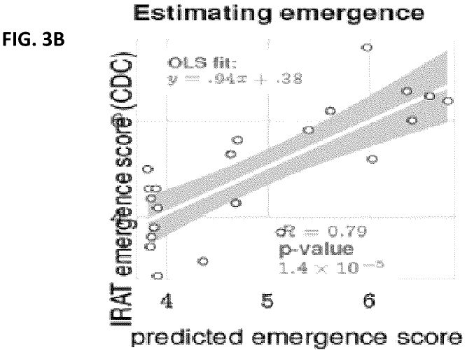

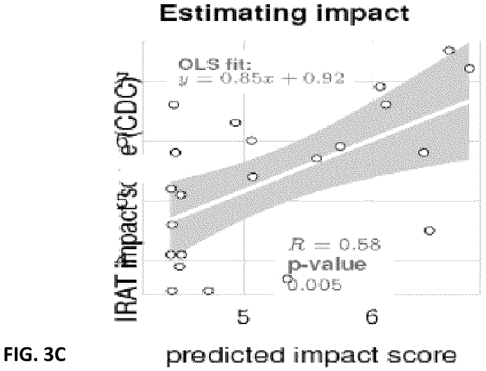

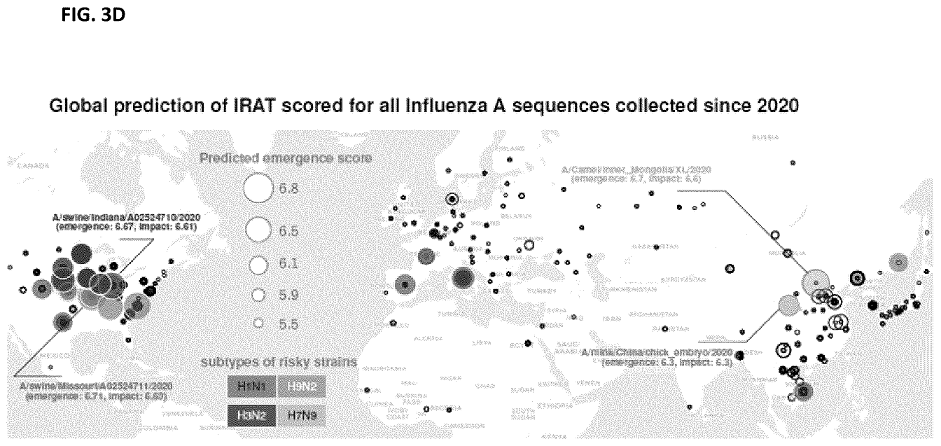

To validate the score against CDC-estimated IRAT emergence scores, Emergenet models were constructed for HA and NA sequences using subtype-specific human strains, typically collected within the year prior to the assessment date, e.g., the assessment date for A/swine/Shandong/1207/2016 is 06/2020, and human H1N1 strains collected between Jan. 7, 2019 and Jun. 30, 2020 were used for the Emergenet inference. For sub-types with less recorded human strains (H1N2, H7N7), all subtype-specific human strains collected up to the assessment date were considered to infer the Emergenet. For subtypes with very few or no recorded human strains even without a lower date bound (H5N2, H5N6, H5N8, H7N8, H9N2, H10N8), the Emergenet was constructed using all human strains that match the HA subtype, e.g., H5Nx for H5N2, H5N6, and H5N8. This addresses the general concern that Emergenet may not be able assess the threat posed by the viruses that have yet to be detected in sufficient numbers; the strains for which this method (marked by ** in Supplementary Table 1) was fit along the fit line in FIG. 3A. The average E-risk for both HA and NA sequences (using Eq. (3)) was then computed. This process was repeated ten times for each strain, taking 100 random sequence samples from the human population in each repetition, finally reporting the average negative geometric mean of the ten HA and NA E-risk iterations as the estimated risk for the strain.

FIG. 3A illustrates a calculated approximate linear relationship between the average E-distance from human circulating strains (negative geometric mean of the E-distance for HA and NA sequences) and the published IRAT emergence scores calculated by the CDC. FIG. 3B illustrates an estimation of the IRAT emergence score via fitting a GLM model to the E-distances estimated from the Emergenet. FIG. 3C illustrates an estimate of IRAT impact scores via fitting a separate GLM model to the E-distances. FIG. 3D illustrates a global prediction of IRAT scored for all influenza A sequences collected since 2020, identifying risky Influenza A strains amongst those collected between January 2020 and September 2022 using the Emergenet approach.

Considering IRAT emergence scores of 22 strains published by the CDC, strong out-of-sample support (correlation of 0.707, pvalue <0.00024, FIG. 3A-D) is found for this claim. Importantly, each E-risk score is computable in approximately 6 seconds as opposed to potentially weeks taken by IRAT experimental assays and SME evaluation. Additionally, using a subtype-specific Emergenet modulates the metric of comparison of genomic sequences, adapting it to the specific subtype of the virus. The time-dependence of the E-risk reflects the impact of the changing background, and recomputing the risk estimates using Emergenets constructed from the recent circulating strains instead of using those from when the IRAT assessments took place at the CDC, worsens the correlation (0.601, p-value 0.003, see Supplementary Table 8). Note that the nature of risk implies that a “high-risk” strain is not guaranteed to emerge, and neither IRAT nor the assessment should be dismissed on the basis that a high-risk strain did not cause a pandemic. However, several instances where IRAT predictions have broadly corroborated with outbreaks are still highlighted. (Supplementary Table 7).

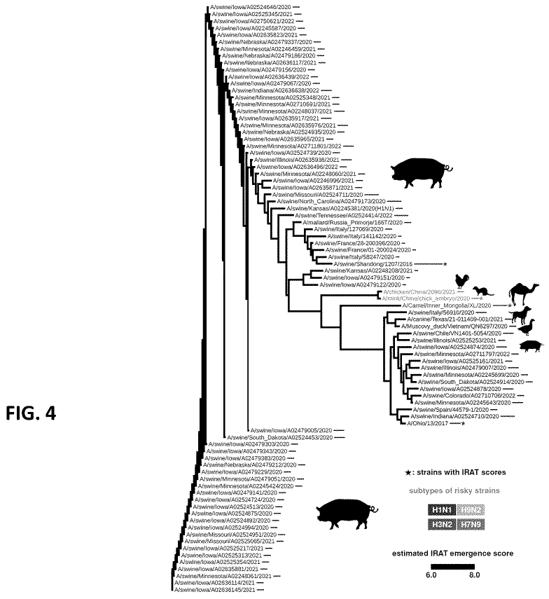

To map the Emergenet distances to more recognizable IRAT scores, a general linear model (GLM) was trained from the HA/NA-based E-risk values (Multivariate Regression to Identify Map from E-distance to Esti-mated IRAT scores, Methods and Supplementary Table 4). Since the CDC-estimated IRAT impact scores are strongly correlated with their IRAT emergence scores (correlation of 0.8015), a separate GLM to was also trained to estimate the impact score from the E-risk values (Supplementary Table 5). Finally, the IRAT scores were estimated for all 6,066 Influenza A strains sequenced globally between January 2020 through September 2022. HA and NA Emergenet models were trained for each subtype using recent sequence data, computed E-risk using the same method as described above, and identified the ones posing maximal risk (FIG. 3C). 1,756 strains turn out to have a predicted emergence score >6.0. However, many of these strains are highly similar, differing by only a few edits. To identify the sufficiently distinct risky strains, the standard phylogeny was constructed from HA sequences with score >6.0 (FIG. 4), and collapsed all leaves within 15 edits, showing only the most risky strain within a collapsed group. As shown in FIG. 4, this leaves 75 strains, with 68 having emergence risk >6.25, and 6 with risk above 6.5 (Extended Data Table 10). FIG. 4 illustrates a standard phylogeny constructed with edit distances, with all Influenza A strains collected between January 2020 and September 2022, with estimated IRAT emergence risk >6.0, and top strains which have IRAT scores (not necessarily from that time period, as shown by *). Leaves have been collapsed that differ by less than 15 edits in the HA, displaying the most risky strains in the collapsed group, which comes from diverse animal hosts and geographic regions.

Subtypes of the risky strains are overwhelmingly H1N1, followed by H3N2, with a small number of H7N9 and H9N2. Five maximally risky strains with emergence score >6.58 are identified to be: A/swine/Missouri/A02524711/2020 (H1N1), A/Camel/Inner Mongolia/XL/2020 (H7N9), A/swine/Indiana/A02524710/2020 (H3N2), A/swine/North Carolina/A02479173/2020 (H1N1), and A/swine/Tennessee/A02524414/2022 (H1N1). Additionally, A/mink/China/chick embryo/2020 (H9N2), with a lower estimated emergence score (6.26) is also important, as the most risky H9N2 strain in the analysis. The HA sequences are compared along with two dominant human strains in 2021-2022 season (FIG. 7), which shows substantial residue replacements, in and out of the receptor binding domain (RBD).

Swine are known to be efficient mixing vessels, and hence unsurprisingly host a large fraction of the risky strains (>80% over 6.0, to over 50% over 6.5). Also, as expected, most of these swine strains are of H1N1 subtype, with the other subtypes having emerged into humans more recently. The finding that a H7N9 poses substantial risk is likewise not surprising: HH transmission has been suspected in Asian-lineage H7N9 strains, and are rated by IRAT as having the greatest potential to cause a pandemic. The finding of the most risky H9N2 strain in a mink is also unsurprising, in the light of these hosts been recently suggested as efficient mixing vessels to breed human-compatible strains. Thus, qualitatively the results are well aligned with the current expectations; nevertheless the ability to quantitatively rank specific strains which pose maximal risk is a crucial new capability enabling proactive pandemic mitigation efforts.

### Results

While numerous tools exist for ad hoc quantification of genomic similarity, higher similarity between strains in these frameworks is not sufficient to imply a high likelihood of a jump. The Emergenet algorithm is the first of its kind to learn an appropriate biologically meaningful comparison metric from data, without assuming any model of DNA or amino acid substitution, or a genealogical tree a priori. While the effect of the environment and selection cannot be inferred from a single sequence, an entire database of observed strains, processed through the right lens, can parse out useful predictive models of these complex interactions. These results are aligned with recent studies demonstrating effective predictability of future mutations for different organisms.

The E-distance calculation is currently limited to analogous sequences (such as point variations of the same protein from different viral subtypes), and the Emergenet inference requires a sufficient diversity of observed strains. A multi-variate regression analysis indicates that the most important factor for the approach to succeed is the diversity of the sequence dataset (General linear model for evaluating effect of data diversity on Emergenet performance, Methods and Supplementary Table 6), which would exclude applicability to completely novel pathogens with no related human variants, and ones that evolve very slowly. Nevertheless, the tools reported here can improve effectiveness of the annual flu shot, and perhaps allow for the development of preemptive vaccines to target risky animal strains before the first human infection in the next pandemic. Apart from outlining new precision public health measures to avert pandemics, such strategies might also help to non-controversially counter the impact of vaccine hesitancy which has interfered with optimal pandemic response in recent times.

### Methods

### Emergenet Framework

It is not assumed that the mutational variations at the individual indices of a genomic sequence are independent (See FIG. 1A). Irrespective of whether mutations are truly random, since only certain combinations of individual mutations are viable, individual mutations across a genomic sequence replicating in the wild appear constrained, which is what is explicitly modeled in the approach.

Consider a set of random variables X={Xᵢ} with i∈{1, . . . , N}, each taking value from the respective sets Σᵢ. Here, each Xᵢ is the random variable modeling the “outcome” i.e. the AA residue at the i^{th} index of the protein sequence. A sample x∈Π₁^{N}Σᵢ is an ordered N-tuple, which is a specific strain in this context, consisting of a realization of each of the variables Xᵢ with the i^{th} entry xᵢ being the realization of random variable Xᵢ.

The notation x₋ᵢ and x^{i,σ} is used to denote:

$${x}_{- i} {=}^{Δ} {x}_{1} , … {x}_{i - 1} , {x}_{i + 1} , … , {x}_{N} \qquad ( 4 a )$$

$${x}^{i , σ} {=}^{Δ} {x}_{1} , … {x}_{i - 1} , σ , {x}_{i + 1} , … , {x}_{N} , σ ∈ {Σ}_{i} \qquad ( 4 b )$$

Also, [symbol] (S) denotes the set of probability measures on a set S, e.g., [symbol] (Σᵢ) is the set of distributions on Σᵢ. It is noted that X defines a random field over the index set {1, . . . , N}.

Definition 1 (Emergenet). For a random field X={Xᵢ} indexed by i∈{1, . . . , N}, the Emergenet is defined to be the set of predictors ϕ={ϕᵢ}, i.e., the following applies:

$${Φ}_{i} = {\prod }_{j \neq i} {Σ}_{j} \to 𝒟 ( {Σ}_{i} ) \qquad ( 5 )$$

where for a sequence x, ϕᵢ(x₋₁) estimates the distribution of Xᵢ on the set Σᵢ.

Conditional inference trees are used as models for predictors, although more general models are possible.

### Biology-Aware Distance Between Sequences

The mathematical form of the metric is not arbitrary; JS divergence is a symmetricised version of the more common KL divergence between distributions, and among different possibilities, the E-distance is the simplest metric such that the likelihood of a spontaneous jump (See Eq. (9) in Methods) is provably bounded above and below by simple exponential functions of the E-distance.

Definition 2 (E-distance: adaptive biologically meaningful dissimilarity between sequences). Given two sequences x, y∈Π₁^{N}Σᵢ, such that when x, y are drawn to from the populations P,Q inducing the Emergenet ϕ^{P}, ϕ^{Q}, respectively, a pseudo-metric θ(x,y) is defined as follows:

$$θ ( x , y ) {=}^{Δ} {E}_{i} ( {𝕁}^{\frac{1}{2}} ( {Φ}_{i}^{P} ( {x}_{- i} ) , {Φ}_{i}^{Q} ( {y}_{- i} ) ) ) \qquad ( 6 )$$

where [symbol] (⋅,⋅) is the Jensen-Shannon divergence and Eᵢ indicates expectation over the indices.

The square root in the definition arises naturally from the bounds that are able to be proven, and is dictated by the form of Pinsker's inequality, ensuring that the sum of the length of successive path fragments equates the length of the path.

### Membership Degree

For modeling to be reliable, a quantitative test of how well the Emergenet represents the data is needed. Here, an explicit membership test is formulated to ascertain if individual samples may indeed be generated by the Emergenet with sufficiently high probability.

Definition 3 (Membership probability of a sequence). Given a population P inducing the Emergenet ϕ^{P} and a sequence x, one can compute the membership probability of x:

$${ω}_{x}^{P} {=}^{Δ} Pr ( x ∈ P ) = {\prod }_{j = 1}^{N} ( {Φ}_{j}^{P} ( {x}_{- j} ) \vert {x}_{j} ) \qquad ( 7 )$$

where xⱼ is the j^{th} entry in x, and is thus an element in the set Σⱼ. Since a main concern is the case where Σⱼ is a finite set, ϕⱼ^{P}(x₋ⱼ)|xⱼ is the entry in the probability mass function corresponding to the element of Σⱼ which appears at the j^{th} entry in sequence x.

This calculation can be carried out for a sequence x known to be in the population P as well, which allows us to define the membership degree ωₓ^{P}.

Definition 4 (Membership degree). Let X be a random field representing a population P, i.e., X=x is a randomly drawn sequence from P. Then the membership degree ω^{P} is a function of the random variable X:

$${ω}^{P} ( X ) {=}^{Δ} {\prod }_{j = 1}^{N} ( {Φ}_{j}^{P} ( {x}_{j}^{-} ) {\vert}_{{x}_{j}} ) \qquad ( 8 )$$

Note that ω^{P} takes values in the unit interval [0,1] and the probability x is a member of the population P is ω^{P}(X=x), denoted as ωₓ^{P} or ωₓ if P is clear from context.

Since ω^{P}(X) is a random variable, sets of sequences can now be computed that better represent the population P, and ones that are on the fringe. Whether a particular sequence is not from the population P can now be evaluated using a pre-specified significance level.

### Theoretical Probability Bounds

The Emergenet framework allows for the rigorous computation of bounds on the probability of a spontaneous change of one strain to another, brought about by chance mutations. While any sequence of mutations is equally likely, the “fitness” of the resultant strain, or the probability that it will even result in a viable strain, or not. Thus, the necessity of preserving function dictates that not all random changes are viable, and the probability of observing some trajectories through the sequence space are far greater than others. The Emergenet framework allows for the exploration of this constrained dynamics, as revealed by a sufficiently large set of genomic sequences.

The mathematical intuition relating E-distance to the log-likelihood of spontaneous change is similar to quantifying the odds of a rare biased outcome when tossing a fair coin. While for an unbiased coin, the odds of roughly 50% heads are overwhelmingly likely, large deviations do happen rarely, and it turns out that the probability of such rare deviations can be explicitly quantified with existing statistical theory. Generalizing to non-uniform conditional distributions inferred by the Emergenet, the likelihood of a spontaneous transition by random chance may also be similarly bounded.

It is shown in Theorem 1 in the supplementary text (Eq. (9)) that at a significance level α, with a sequence length N, the probability of spontaneous jump of sequence x from population P to sequence y in population Q, Pr(x→y), is bounded by

$${ω}_{y}^{Q} {e}^{\frac{\sqrt{8} {N}^{2}}{1 - α} θ ( x , y )} ≧ P r ( x \to y ) ≧ {ω}_{y}^{Q} {e}^{- \frac{\sqrt{8} {N}^{2}}{1 - α} θ ( x , y )} \qquad ( 9 )$$

where ω_{y}^{Q} is the membership probability of strain y in the target population, N is the sequence length, and α is the statistical significance level.

### Predicting Future Dominant Seasonal Strains

Analyzing the distribution of sequences observed to circulate in the human population at the present time allows for the forecast of dominant strain(s) in the next flu season as follows:

Let x_{*}^{t+δ} be a dominant strain in the upcoming flu season at time t+δ, where H^{t} is the set of observed strains presently in circulation in the human population (at time t). It is assumed that the Emergenet is constructed using the sequences in the set H^{t}, and remains unchanged up to t+δ. Since this set is a function of time, the inferred Emergenet also changes with time, and the induced E-distance is denoted as θ^{[t]}.

From the RHS bound established in Theorem 1 (See Eq. (9) above) in the supplementary text, the following applies:

$$ln \frac{P r ( x \to {x}^{t + α} )}{{ω}_{{x}^{t + δ}}} ≧ - \frac{\sqrt{8} {N}^{2}}{1 - α} {θ}^{[ t ]} ( x , {x}^{t + δ} ) \qquad ( 10 )$$

$$⇒ {\sum }_{x ∈ {H}^{t}} ln \frac{P r ( x \to {x}^{t + α} )}{{ω}_{{x}^{t + δ}}} ≧ {\sum }_{x ∈ {H}^{t}} - \frac{\sqrt{8} {N}^{2}}{1 - α} {θ}^{[ t ]} ( x , {x}^{t + δ} ) \qquad ( 11 )$$

$$⇒ {\sum }_{x ∈ {H}^{t}} {θ}^{[ t ]} ( x , {x}^{t + δ} ) - \vert {H}^{t} \vert A ln {ω}_{{x}^{t + δ}} ≧ A ln \frac{1}{{\prod }_{x ∈ {H}^{t}} P r ( x \to {x}^{t + δ} )} \qquad ( 12 )$$

where

$$A = \frac{1 - α}{\sqrt{8} {N}^{2}} ,$$

where N is the equated significance level. Since minimizing the LHS maximizes the lower bound on the probability of the observed strains simultaneously giving rise to x^{t+δ}, a dominant strain x_{*}^{t+δ} may be estimated as a solution to the optimization problem:

$${x}_{*}^{t + δ} = \begin{matrix}arg min \\ y ∈ {⋃}_{r \leq t} {H}^{r}\end{matrix} ( {\sum }_{x ∈ {H}^{t}} {θ}^{[ t ]} ( x , y ) - \vert {H}^{t} \vert A ln {ω}_{y} \qquad ( 13 )$$

In Eq. (13), the assumption that H^{t} is a single cluster of strains is made. In practice, this may not be the case; it is possible for several distinct clusters to arise in each season. The strains can be clustered by using a clustering method (such as MeanShift) on the E-distance matrix computed between strains in H^{t} such that there are n disjoint clusters H₁^{t}, H₂^{t}, . . . Hₙ^{t}, with ∪ᵢ₌₁ⁿHᵢ^{t}=H^{t}. Thus, multiple predictions can be made per season, by replacing H^{t} in Eq. (13) with Hᵢ^{t} for i=1, 2, . . . n. In some embodiments, the predictions yielded from the largest to clusters are taken.

Measure of Pandemic Potential (Bottom of p11)

The potential of an animal strain x_{α}^{t} to spillover and become HH capable as a human strain xₕ^{t+δ} is measured via the proposed E-risk defined as follows:

$$ρ ( {x}_{α}^{t} ) {=}^{Δ} - \frac{1}{\vert {H}^{t} \vert} {\sum }_{x ∈ {H}^{t}} {θ}^{[ t ]} ( {x}_{a}^{t} , x ) \qquad ( 14 )$$

whereas before H^{t} is the set of human strains observed recently (taken as strains collected within the past year), and θ^{[t]} is the E-distance induced by the Emergenet computed from the sequences in H^{t}.

The intuition here is that a lower bound of ρ(x_{α}^{t}) scales as average log-likelihood of the x_{α}^{t} giving rise to a human strain in circulation at time t. Since the strains in H^{t} are already HH capable, a high average likelihood of producing a similar strain has a high potential of being a HH cable novel variant, which is a necessary condition of a pandemic strain. To establish the lower bound, from Theorem 1 (See Eq. (9) above) in the supplementary text:

$${\sum }_{y ∈ {H}^{t}} ln \vert \frac{P r ( {x}_{a}^{t} \to y )}{{ω}_{y}} \vert ≦ - \frac{\sqrt{\{ 8 \} {N}^{2}}}{1 - α} \vert {H}^{t} \vert ρ ( {x}_{a}^{t} ) \qquad ( 15 )$$

### Denoting

$$A = \frac{1 - α}{\sqrt{\{ 8 \} {N}^{2}}} ,$$

A ln Π_{y∈H}_{^{t}}ω_{y}=C and <⋅> as the geometric mean function, this leads to:

$$⇒ ρ ( {x}_{α}^{t} ) ≧ A ln ( {\prod }_{y ∈ {H}^{t}} Pr ( {x}_{α}^{t} \to y ) + C \qquad ( 16 )$$

$$⇒ ρ ( {x}_{α}^{t} ) ≧ A ln 〈 Pr ( {x}_{α}^{t} \to {x}_{h}^{t + δ} ) 〉 + C \qquad ( 17 )$$

By noting that A, C, are not functions of x_{α}^{t}, it can be concluded that a lower bound of the proposed risk measure ρ(⋅) scales with the average loglikelihood of producing strains close to a circulating human strain at the current time.

### Proof of Probability Bounds

Theorem 1 (Probability bound). Given a sequence x of length N that transitions to a strain y∈Q, the following bounds are found at significance level α.

$${ω}_{y}^{Q} {e}^{\frac{\sqrt{8} {N}^{2}}{1 - α} θ ( x , y )} ≧ Pr ( x \to y ) ≧ {ω}_{y}^{Q} {e}^{- \frac{\sqrt{8} {N}^{2}}{1 - α} θ ( x , y )} \qquad ( 18 )$$

where ω_{y}^{Q} is the membership probability of strain y in the target population Q (see Def. 3), and θ(x,y) is the q-distance between x,y (see Def. 2).

Proof. Using Sanov's theorem on large deviations, it can be concluded that the probability of spontaneous jump from strain x∈P to strain y∈Q with the possibility P≠Q, is given by:

$${Pr ( x \to y ) = {\prod }_{i = 1}^{N} ( {Φ}_{i}^{P} ( {x}_{- i} ) \vert}_{{y}_{i}} ) \qquad ( 19 )$$

Writing the factors on the right hand side as:

$${{{Φ}_{i}^{P} ( {x}_{- i} ) \vert}_{{y}_{i}} = {Φ}_{i}^{Q} ( {y}_{- i} ) \vert}_{{y}_{i}} ( \frac{{{Φ}_{i}^{P} ( {x}_{- i} ) \vert}_{{y}_{i}}}{{{Φ}_{i}^{Q} ( {y}_{- i} ) \vert}_{{y}_{i}}} ) \qquad ( 20 )$$

it is noted that ϕᵢ^{P}(x₋ᵢ),ϕᵢ^{Q}(y₋ᵢ) are distributions on the same index i, and hence:

$$\vert {{Φ}_{i}^{P} ( {x}_{- i} )}_{{y}_{i}} - {{Φ}_{i}^{Q} ( {y}_{- i} )}_{{y}_{i}} \vert ≦ {\sum }_{y ∈ {\sum }_{i}} \vert {{Φ}_{i}^{P} ( {x}_{- i} )}_{{y}_{i}} - {{Φ}_{i}^{Q} ( {y}_{- i} )}_{{y}_{i}} \vert \qquad ( 21 )$$

Using a standard refinement of Pinsker's inequality, and the relationship of Jensen-Shannon divergence with total variation:

$${θ}_{i} ≧ \frac{1}{8} {\vert {{Φ}_{i}^{P} ( {x}_{- i} )}_{{y}_{i}} - {{Φ}_{i}^{Q} ( {y}_{- i} )}_{{y}_{i}} \vert}^{2} ⇒ \vert 1 - \frac{{{Φ}_{i}^{Q} ( {y}_{- i} )}_{{y}_{i}}}{{{Φ}_{i}^{P} ( {x}_{- i} )}_{{y}_{i}}} \vert ≦ \frac{1}{{a}_{0}} \sqrt{8 {θ}_{i}} \qquad ( 22 )$$

where a₀ is the smallest non-zero probability value of generating the entry at any index. This parameter is related to the statistical significance of the bounds. First, the lower bound can be formulated as:

$$log {\prod }_{i = 1}^{N} \frac{{{Φ}_{i}^{P} ( {x}_{- i} ) \vert}_{{y}_{i}}}{{{Φ}_{i}^{Q} ( {y}_{- i} ) \vert}_{{y}_{i}}} = {\sum }_{i} log ( \frac{{{Φ}_{i}^{P} ( {x}_{- i} ) \vert}_{{y}_{i}}}{{{Φ}_{i}^{Q} ( {y}_{- i} ) \vert}_{{y}_{i}}} ) ≧ {\sum }_{i} ( 1 - \frac{{{Φ}_{i}^{P} ( {x}_{- i} ) \vert}_{{y}_{i}}}{{{Φ}_{i}^{Q} ( {y}_{- i} ) \vert}_{{y}_{i}}} ) ≧ \frac{\sqrt{8}}{{a}_{0}} {\sum }_{i} {θ}_{i}^{( \frac{1}{2} )} = - \frac{\sqrt{8} N}{{a}_{0}} θ \qquad ( 23 )$$

Similarly, the upper bound may be derived as:

$$log {\prod }_{i = 1}^{N} \frac{{{Φ}_{i}^{P} ( {x}_{- i} ) \vert}_{{y}_{i}}}{{{Φ}_{i}^{Q} ( {y}_{- i} ) \vert}_{{y}_{i}}} = {\sum }_{i} log ( \frac{{{Φ}_{i}^{P} ( {x}_{- i} ) \vert}_{{y}_{i}}}{{{Φ}_{i}^{Q} ( {y}_{- i} ) \vert}_{{y}_{i}}} ) ≦ {\sum }_{i} ( \frac{{{Φ}_{i}^{P} ( {x}_{- i} ) \vert}_{{y}_{i}}}{{{Φ}_{i}^{Q} ( {y}_{- i} ) \vert}_{{y}_{i}}} - 1 ) ≦ \frac{\sqrt{8} N}{{a}_{0}} θ \qquad ( 24 )$$

Eqs. (23) and (24) can be combined to conclude:

$${ω}_{y}^{Q} {e}^{\frac{\sqrt{8} {N}^{2}}{1 - α} θ} ≧ Pr ( x \to y ) ≧ {ω}_{y}^{Q} {e}^{- \frac{\sqrt{8} {N}^{2}}{1 - α} θ} \qquad ( 25 )$$

Now, interpreting α₀ as the probability of generating an unlikely event below the desired threshold (i.e., a “failure”), it can be noted that the probability of generating at least one such event is given by 1−(1−a₀)^{N}. Hence, if α is the pre-specified significance level, for N>>1:

$${a}_{0} \approx ( 1 - α ) / N \qquad ( 26 )$$

Hence, it is concluded that at significance level≥α, the bounds are:

$${ω}_{y}^{Q} {e}^{\frac{\sqrt{8} {N}^{2}}{1 - α} θ} ≧ Pr ( x \to y ) ≧ {ω}_{y}^{Q} {e}^{- \frac{\sqrt{8} {N}^{2}}{1 - α} θ} \qquad ( 27 )$$

This bound can be rewritten in terms of the log-likelihood of the spontaneous jump and constants independent of the initial sequence x as:

$$\vert log Pr ( x \to y ) - {C}_{0} \vert ≦ {C}_{1} θ \qquad ( 28 )$$

where the constants are given by:

$${C}_{0} = log {ω}_{y}^{Q} \qquad ( 29 )$$

$${C}_{1} = \frac{\sqrt{8} {N}^{2}}{1 - α} \qquad ( 30 )$$

### In-Silico Corroboration of Emergenet's Capability to Capture Biologically Meaningful Structure

The results of simulated mutational perturbations are compared to sequences from databases (for which Emergenets are constructed), and then used NCBI BLAST (https://blast.ncbi.nlm.nih.gov/Blast.cgi) to identify if the perturbed sequences match with existing sequences in the databases (FIGS. 8A-E). In contrast to random variations, which rapidly diverge the trajectories, the Emergenet constraints tend to produce smaller variance in the trajectories, maintain a high degree of match as the trajectories are extended, and produces matches closer in time to the collection time of the initial sequence, suggesting that the Emergenet does indeed capture realistic constraints.

FIGS. 8A-E illustrates that the Emergenet induced modeling of evolutionary trajectories initiated from known haemagglutinnin (HA) sequences are distinct from random paths in the strain space. In particular, random trajectories have more variance, and more importantly, diverge to different regions of the landscape compared to Emergenet predictions in FIG. 8A. In FIGS. 8B-E, unconstrained Q-sampling produces sequences are shown to maintain a higher degree of similarity to known sequences, as verified by blasting against known HA sequences, have a smaller rate of growth of variance, and produce matches in closer time frames to the initial sequence. FIG. 8C shows that this is not due to simply restricting the mutational variations, which increases rapidly in both the Emergenet and the classical metric.

### Multivariate Regression to Understand Data Characteristics Necessary for Emergenet Modeling

The key factors that contribute to modeling a set of strains well within the Emergenet framework were investigated. A multivariate regression was carried out with data diversity, the complexity of inferred Emergenet and the edit distance of the WHO recommendation from the dominant strain as independent variables (See Supplementary Table 6 for definitions). Here data diversity is defined as the number of clusters in the input set of sequences, such that any two sequences five or less mutations apart are in the same cluster. Emergenet complexity is measured by the number of decision nodes in the component decision trees of the recursive forest.

Several plausible structures of the regression equation are selected, and in each case it is concluded that data diversity has the most important and statistically significant contribution (Supplementary Table 6).

Multivariate Regression to Identify Map from E-Distance to Estimated IRAT Scores

Separate General Linear Models (GLM) are trained to estimate IRAT scores (emergence and impact) with average E-distance of a sequence of interest from a set of human strains, considering HA and NA sequences separately, using the CDC computed IRAT scores as the dependent variables. The geometric mean of the HA and NA based E-distances are also included as potential explanatory variables. A standard Gaussian model family with identity link function is used to keep the model that maps E-distances to the IRAT scores as simple as possible (see Supplementary Table 4).

### Data and Software Sharing

Working open-source software (requiring Python 3.x) is publicly available at https://pypi.org/project/emergenet/. All inferred Emergenet models inferred is available at https://doi.org/10.5281/zenodo.7387861.

### Data Source

Two public sequence databases are used: 1) National Center for Biotechnology Information (NCBI) virus and 2) GISAID databases. The former is a community portal for viral sequence data, aiming to increase the usability of data archived in various NCBI repositories. GISAID has a more restricted user agreement, and use of GISAID data in an analysis requires acknowledgment of the contributions of both the submitting and the originating laboratories (Corresponding acknowledgment tables are included as supplementary information). A total of 405,778 sequences were collected in analysis (see Supplementary Table 1).

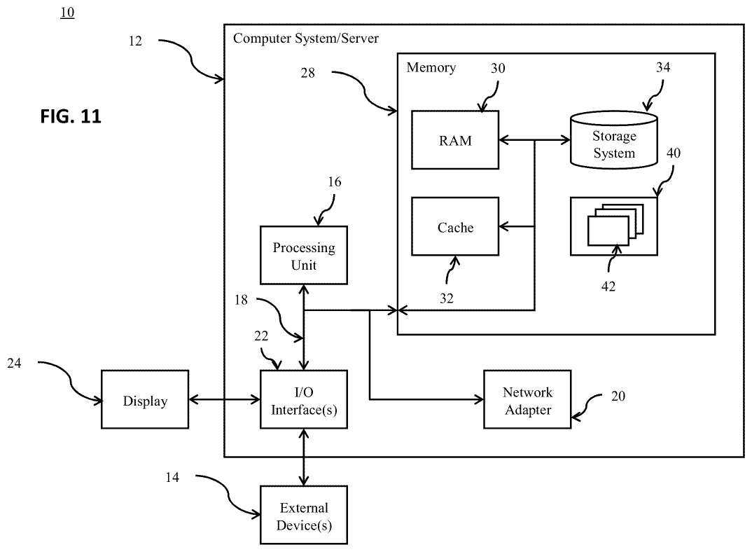

Referring now to FIG. 11, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 11, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Extended Data Table 1
Influenza A Strains Evaluated by IRAT and Corresponding Emergenet Computed Risk Scores

| | | | IRAT | | | | | Enet | Enet | | |
|---|---|---|---|---|---|---|---|---|---|---|---|
| | | | Emer- | IRAT | | | [symbol] | Emer- | Emer- | Enet | Enet |
| | | IRAT | gence | Impact | HA | NA | Mean | gence | gence | Impact | Impact |
| Influenza Virus | [symbol] | [symbol] | Score | Score | Sample | Sample | E-risk | Score | Err. | Score | Err. |
| A/swine/ [symbol] | H1N1 | July 2020 | 7.5 | [symbol] | 8583 | 8583 | [symbol] | [symbol] | 0.0020 | [symbol] | 0.0017 |
| A/ [symbol] | H3N2 | July 2015 | [symbol] | [symbol] | [symbol] | 12388 | [symbol] | [symbol] | 0.0059 | [symbol] | 0.0052 |
| A/Hong Kong/125/2017 | [symbol] | May 2017 | [symbol] | 7.5 | [symbol] | 437 | [symbol] | [symbol] | [symbol] | [symbol] | [symbol] |
| A/Shanghai/02/2013 | [symbol] | April 2016 | [symbol] | 7.2 | 178 | 178 | [symbol] | [symbol] | [symbol] | [symbol] | [symbol] |
| A/ [symbol] | H9N2 | July 2019 | [symbol] | 5.9 | [symbol] | [symbol] | [symbol] | [symbol] | 0.1957 | 5.7449 | [symbol] |
| A/Indiana/ [symbol] /2011 | H3N2 | December 2012 | [symbol] | 4.5 | 2298 | 2298 | 0.0210 | [symbol] | 0.0320 | [symbol] | [symbol] |
| A/California/ [symbol] | [symbol] | July 2019 | 5.8 | [symbol] | 55 | [symbol] | [symbol] | [symbol] | [symbol] | [symbol] | [symbol] |
| A/ [symbol] | [symbol] | February 2014 | [symbol] | [symbol] | [symbol] | [symbol] | [symbol] | [symbol] | [symbol] | [symbol] | [symbol] |
| A/ [symbol] | [symbol] | October 2021 | 5.3 | 6.3 | 45 | 45 | [symbol] | [symbol] | [symbol] | [symbol] | [symbol] |
| A/Vietnam/ [symbol] | [symbol] | November 2011 | 5.2 | [symbol] | 257 | 243 | [symbol] | [symbol] | [symbol] | [symbol] | [symbol] |
| A/ [symbol] | [symbol] | April 2016 | [symbol] | [symbol] | 344 | [symbol] | 0.3211 | 3.8000 | [symbol] | [symbol] | [symbol] |
| A/ [symbol] | [symbol] | March 2021 | [symbol] | [symbol] | [symbol] | [symbol] | [symbol] | [symbol] | 0.0151 | [symbol] | [symbol] |
| A/ [symbol] | [symbol] | June 2012 | [symbol] | [symbol] | [symbol] | [symbol] | [symbol] | [symbol] | [symbol] | [symbol] | [symbol] |
| A/American [symbol] / | [symbol] | March 2022 | [symbol] | 5.1 | 334 | 317 | [symbol] | [symbol] | [symbol] | [symbol] | [symbol] |
| South Carolina/AH [symbol] | | | | | | | | | | | |
| A/ [symbol] | [symbol] | February 2014 | [symbol] | 6.0 | [symbol] | [symbol] | [symbol] | 4.6710 | [symbol] | [symbol] | [symbol] |
| A/ [symbol] /Washington/ | [symbol] | March 2015 | [symbol] | [symbol] | 341 | 328 | [symbol] | [symbol] | 0.0157 | [symbol] | [symbol] |
| [symbol] | | | | | | | | | | | |
| A/Northern [symbol] /Washington/ | [symbol] | March 2015 | 3.8 | 4.1 | [symbol] | 3.28 | 0.2416 | 3.8550 | 0.0115 | 4.4257 | 0.0012 |
| [symbol] | | | | | | | | | | | |
| A/ [symbol] /Illinois/ [symbol] /2015 | H3N2 | June 2016 | 3.7 | 3.7 | 4180 | 4180 | [symbol] | [symbol] | 0.0025 | 5.3315 | 0.0021 |
| A/American green-winged | [symbol] | March 2015 | [symbol] | [symbol] | 325 | [symbol] | [symbol] | [symbol] | 0.0155 | [symbol] | [symbol] |
| [symbol] /Washington/ [symbol] /2014 | | | | | | | | | | | |
| A/turkey/Indiana/ [symbol] | [symbol] | July 2017 | 3.4 | [symbol] | [symbol] | [symbol] | [symbol] | [symbol] | [symbol] | [symbol] | [symbol] |
| A/chicken/Tennessee/ [symbol] | [symbol] | October 2017 | 3.1 | 3.5 | [symbol] | [symbol] | [symbol] | [symbol] | 0.0507 | [symbol] | [symbol] |
| [symbol] | | | | | | | | | | | |
| A/chicken/Tennessee/ [symbol] | [symbol] | October 2017 | 2.8 | 3.5 | [symbol] | [symbol] | [symbol] | [symbol] | 0.0045 | [symbol] | [symbol] |
| [symbol] | | | | | | | | | | | |

[symbol]

indicates data missing or illegible when filed

Extended Data Table 2
Out-performance of Emergenet Recommendations over WHO for Influenza A Vaccine Composition

| | | | Two Decades | | | One Decade | | | 2015-2019 | | |
|---|---|---|---|---|---|---|---|---|---|---|---|
| | | | WHO | Enet | Improve- | WHO | Enet | Improve- | WHO | Enet | Improve- |
| Subtype | Gene | Hemisphere | Error | Error | ment (%) | Error | Error | ment (%) | Error | Error | ment (%) |
| H1N1 | HA | North | 14.54 | 9.76 | 48.97 | 10.39 | 4.14 | 150.88 | 10.10 | 3.81 | 164.67 |
| H1N1 | HA | South | 13.67 | 8.70 | 57.17 | 10.28 | 4.02 | 155.83 | 9.69 | 3.80 | 154.71 |
| H1N1 | HA | Average | 14.10 | 9.23 | 52.83 | 10.34 | 4.08 | 153.32 | 9.89 | 3.81 | 159.70 |
| H3N2 | HA | North | 9.10 | 5.53 | 64.58 | 8.76 | 6.40 | 36.74 | 8.08 | 5.94 | 35.97 |
| H3N2 | HA | South | 8.51 | 4.83 | 76.38 | 8.11 | 5.16 | 57.19 | 7.27 | 4.08 | 77.94 |
| H3N2 | HA | Average | 8.81 | 5.18 | 70.08 | 8.43 | 5.78 | 45.86 | 7.68 | 5.01 | 53.06 |
| H1N1 | NA | North | 10.13 | 7.64 | 32.70 | 6.99 | 3.64 | 91.83 | 8.38 | 3.90 | 114.97 |
| H1N1 | NA | South | 10.20 | 7.48 | 36.29 | 6.73 | 3.72 | 81.20 | 7.80 | 4.30 | 81.35 |
| H1N1 | NA | Average | 10.16 | 7.56 | 34.48 | 6.86 | 3.68 | 86.46 | 8.09 | 4.10 | 97.34 |
| H3N2 | NA | North | 5.36 | 3.56 | 50.80 | 5.52 | 3.40 | 62.15 | 7.15 | 4.58 | 56.15 |
| H3N2 | NA | South | 5.46 | 3.56 | 53.45 | 5.17 | 3.47 | 48.87 | 6.48 | 4.16 | 55.58 |
| H3N2 | NA | Average | 5.41 | 3.56 | 52.13 | 5.35 | 3.44 | 55.44 | 6.82 | 4.37 | 55.88 |

Extended Data Table 3
H1N1 Northern Hemisphere

| | | | | Avg. | Avg. | Avg. | Avg. |
|---|---|---|---|---|---|---|---|
| | WHO | Emergenet | Emergenet | HA WHO | HA Enet | NA WHO | NA Enet |
| Season | Reccomendation | Recommendation 1 | Recommendation 2 | Error | Error | Error | Error |
| 2003-04 | A/New Caledonia/20/99 | A/HaNoi/2143/2001 | A/New York/291/2002 | 7.3 | 2.9 | 3.8 | 4.8 |
| 2004-05 | A/New Caledonia/20/99 | A/HaNoi/2546/2002 | A/Ha Noi/2476/2001 | 10.1 | 8.5 | 4.4 | 4.4 |
| 2005-06 | A/New Caledonia/20/99 | A/HaNoi/2253/2001 | A/Ha Noi/2253/2001 | 7.3 | 5.3 | 4.6 | 4.6 |
| 2006-07 | A/New Caledonia/20/99 | A/Malaysia/25531/2003 | A/Yazd/144/2006 | 9.6 | 6.1 | 5.8 | 3.9 |
| 2007-08 | A/Solomon Islands/ | A/New York/1050/2006 | A/Canterbury/106/2004 | 10.2 | 4.0 | 14.5 | 5.7 |
| | 3/2006 | | | | | | |
| 2008-09 | A/Brisbane/59/2007 | A/England/545/2007 | A/Illinois/UR006-018/ | 5.7 | 4.6 | 4.5 | 5.5 |
| | | | 2007 | | | | |
| 2009-10 | A/Brisbane/59/2007 | A/England/557/2007 | A/Peru/3909/2008 | 112.4 | 111.3 | 84.0 | 82.9 |
| 2010-11 | A/California/7/2009 | A/Taiwan/97196/2009 | A/Bogota/WRAIR0088N/ | 5.5 | 3.2 | 2.4 | 0.1 |
| | | | 2009 | | | | |
| 2011-12 | A/California/7/2009 | A/Ayutthaya/568/2009 | A/Korea/94/2009 | 7.5 | 3.5 | 4.1 | 3.1 |
| 2012-13 | A/California/7/2009 | A/Kenya/Kilifi-011/2010 | A/Taiwan/66169/2010 | 11.3 | 4.3 | 4.7 | 1.4 |
| 2013-14 | A/California/7/2009 | A/IIV-Vladimir/67/2011 | A/American_Samoa/4520/ | 11.8 | 2.5 | 8.0 | 3.1 |
| | | | 2011 | | | | |
| 2014-15 | A/California/7/2009 | A/Cruz Alta/LACENRS-129/ | A/Cluj/133922/2013 | 16.1 | 4.8 | 12.8 | 6.2 |
| | | 2012 | | | | | |
| 2015-16 | A/California/7/2009 | A/Laos/I018/2014 | A/USA/VFFSP_UNITHER_{—} | 15.3 | 5.4 | 13.8 | 7.0 |
| | | | 00011/2014 | | | | |
| 2016-17 | A/California/7/2009 | A/Lebanon/1104/2016 | A/Slovenia/1413/15 | 16.2 | 1.7 | 14.1 | 1.5 |
| 2017-18 | A/Michigan/45/2015 | A/Panama/317744/2016 | A/Sweden/39/2015 | 4.4 | 3.4 | 4.0 | 4.0 |
| 2018-19 | A/Michigan/45/2015 | A/Clermont-Ferrand/ | A/Mississippi/09/2018 | 6.2 | 2.2 | 5.1 | 1.1 |
| | | 1873/2017 | | | | | |
| 2019-20 | A/Brisbane/02/2018 | A/Michigan/200/2018 | A/Maryland/12/2019 | 8.4 | 6.4 | 4.9 | 5.9 |
| 2020-21 | A/Guangdong- | A/Mauritius/I-609/2019 | A/Guyane/1882/2019 | 5.1 | 5.7 | 3.6 | 3.5 |
| | Maonan/SWL1536/2019 | | | | | | |
| 2021-22 | A/Victoria/2570/2019 | A/Washington/16/2020 | A/Nordrhein-westfalen/ | 9.9 | 4.8 | 2.3 | 2.6 |
| | | | 7/2020 | | | | |
| 2022-23 | A/Victoria/2570/2019 | A/Netherlands/00475/2020 | A/Bangladesh/9004/2021 | 10.5 | 4.5 | 1.3 | 1.5 |
| 2023-24 | A/Victoria/4897/2022 | A/Valladolid/18/2023 | A/South_Africa/R05753/ | −1 | −1 | −1 | −1 |
| | | | 2022 | | | | |

Avg. error computed as the average minimum edit distance between our two recommendations and the dominant strains, which is defined as the strain closest to the centroid of each cluster in the edit distance metric. The average error is weighted by the sizes of the clusters the dominant strains belong to.

Extended Data Table 4
H1N1 Southern Hemisphere

| | | | | Avg. | Avg. | Avg. | Avg. |
|---|---|---|---|---|---|---|---|
| | WHO | Emergent | Emergent | HA WHO | HA Enet | NA WHO | NA Enet |
| Season | Recommendation | Recommendation 1 | Recommendation 2 | Error | Error | Error | Error |
| 2003 | A/ [symbol] | A/Memphis [symbol] | A/ [symbol] | [symbol] | [symbol] | [symbol] | [symbol] |
| 2004 | A/ [symbol] | A/ [symbol] | A/ [symbol] | 7.9 | [symbol] | [symbol] | [symbol] |
| 2005 | A/ [symbol] | A/ [symbol] | A/ [symbol] | 7.2 | [symbol] | [symbol] | [symbol] |
| 2006 | A/ [symbol] | A/ [symbol] | A/ [symbol] | 9.2 | [symbol] | 5.8 | [symbol] |
| 2007 | A/ [symbol] | A/ [symbol] | A/ [symbol] | [symbol] | [symbol] | [symbol] | [symbol] |
| 2008 | A/Soloman Island/ [symbol] | A/ [symbol] | A/ [symbol] | [symbol] | [symbol] | [symbol] | [symbol] |
| 2009 | A/Brisbane [symbol] | A/England [symbol] | A/ [symbol] | [symbol] | [symbol] | 74.2 | [symbol] |
| 2010 | A/California [symbol] | A/ [symbol] | A/ [symbol] | [symbol] | [symbol] | [symbol] | [symbol] |
| 2011 | A/California [symbol] | A/ [symbol] | A/ [symbol] | 7.0 | [symbol] | [symbol] | [symbol] |
| 2012 | A/California [symbol] | A/Sydney [symbol] | A/Hong_Kong [symbol] | 10.2 | [symbol] | [symbol] | 2.2 |
| 2013 | A/California [symbol] | A/ [symbol] | A/ [symbol] | 11.2 | [symbol] | 5.7 | [symbol] |
| 2014 | A/California [symbol] | A/ [symbol] | A/ [symbol] | [symbol] | [symbol] | [symbol] | [symbol] |
| 2015 | A/California [symbol] | A/SYDNEY [symbol] | A/ [symbol] | [symbol] | [symbol] | 12.7 | [symbol] |
| 2016 | A/California [symbol] | A/ [symbol] | A/ [symbol] | [symbol] | [symbol] | [symbol] | 9.2 |
| 2017 | A/Michigan [symbol] | A/England [symbol] | A/ [symbol] | [symbol] | [symbol] | [symbol] | [symbol] |
| 2018 | A/Michigan [symbol] | A/Dominican_Republic [symbol] | A/ [symbol] | [symbol] | [symbol] | [symbol] | [symbol] |
| 2019 | A/Michigan [symbol] | A/ [symbol] | A/ [symbol] | [symbol] | [symbol] | [symbol] | [symbol] |
| 2020 | A/Brisbane [symbol] | A/ [symbol] | A/ [symbol] | [symbol] | [symbol] | [symbol] | [symbol] |
| 2021 | A/Victoria [symbol] | A/ [symbol] | A/ [symbol] | [symbol] | [symbol] | [symbol] | [symbol] |
| 2022 | A/Victoria [symbol] | A/ [symbol] | A/ [symbol] | [symbol] | [symbol] | [symbol] | [symbol] |
| 2323 | A/Sydney [symbol] | A/ [symbol] | A/ [symbol] | −1 | −1 | −1 | −1 |

Avg. error computed as the average minimum edit distance between our two recommendations and the dominant strains, which is defined as the strain closest to the centroid of each cluster in the edit distance metric. The average error is weighted by the sizes of the clusters the dominant strains belong to. [symbol]
indicates data missing or illegible when filed

Extended Data Table 5
H3N2 Northern Hemisphere

| | | | | Avg. | Avg. | Avg. | Avg. |
|---|---|---|---|---|---|---|---|
| | WHO | Emergent | Emergent | HA WHO | HA Enet | NA WHO | NA Enet |
| Season | Recommendation | Recommendation 1 | Recommendation 2 | Error | Error | Error | Error |
| 2003-04 | A/Moscow [symbol] | A/ [symbol] | A/ [symbol] | 25.0 | 5.4 | [symbol] | [symbol] |
| 2004-05 | A/ [symbol] | A/ [symbol] | A/ [symbol] | 8.8 | 3.5 | 4.3 | 2.4 |
| 2005-06 | A/California [symbol] | A/ [symbol] | A/ [symbol] | 12.1 | 4.3 | [symbol] | 5.5 |
| 2006-07 | A/Wisconsin [symbol] | A/ [symbol] | A/ [symbol] | [symbol] | [symbol] | [symbol] | [symbol] |
| 2007-08 | A/Wisconsin [symbol] | A/ [symbol] | A/ [symbol] | 8.7 | 4.9 | 7.8 | [symbol] |
| 2008-09 | A/ [symbol] | A/ [symbol] | A/ [symbol] | [symbol] | [symbol] | [symbol] | [symbol] |
| 2009-10 | A/ [symbol] | A/ [symbol] | A/ [symbol] | [symbol] | 5.2 | [symbol] | [symbol] |
| 2010-11 | A/ [symbol] | A/ [symbol] | A/ [symbol] | [symbol] | [symbol] | [symbol] | [symbol] |
| 2011-12 | A/ [symbol] | A/ [symbol] | A/ [symbol] | 8.9 | 3.0 | 5.3 | 3.4 |
| 2012-13 | A/Victoria [symbol] | A/ [symbol] | A/ [symbol] | [symbol] | [symbol] | [symbol] | [symbol] |
| 2013-14 | A/Victoria [symbol] | A/ [symbol] | A/ [symbol] | [symbol] | [symbol] | [symbol] | 2.0 |
| 2014-15 | A/Texas [symbol] | A/ [symbol] | A/ [symbol] | [symbol] | 2.8 | [symbol] | 3.4 |
| 2015-16 | A/Switzerland [symbol] | A/ [symbol] | A/ [symbol] | [symbol] | [symbol] | [symbol] | 2.1 |
| 2016-17 | A/ [symbol] | A/ [symbol] | A/ [symbol] | 5.0 | [symbol] | 8.9 | [symbol] |
| 2017-18 | A/ [symbol] | A/ [symbol] | A/ [symbol] | 5.3 | 5.0 | [symbol] | 2.7 |
| 2018-19 | A/ [symbol] | A/ [symbol] | A/ [symbol] | [symbol] | [symbol] | 8.0 | 7.0 |
| 2019-20 | A/ [symbol] | A/England [symbol] | A/ [symbol] | [symbol] | [symbol] | [symbol] | 4.8 |
| 2020-21 | A/ [symbol] | A/ [symbol] | A/ [symbol] | [symbol] | [symbol] | 4.2 | 0.3 |
| 2021-22 | A/ [symbol] | A/ [symbol] | A/ [symbol] | 15.2 | [symbol] | 2.5 | 2.2 |
| 2022-23 | A/ [symbol] | A/ [symbol] | A/ [symbol] | [symbol] | [symbol] | [symbol] | [symbol] |
| 2023-24 | A/ [symbol] | A/ [symbol] | A/ [symbol] | −1 | −1 | −1 | −1 |

Avg. error computed as the average minimum edit distance between our two recommendations and the dominant strains, which is defined as the strain closest to the centroid of each cluster in the edit distance metric. The average error is weighted by the sizes of the clusters the dominant strains belong to. [symbol]
indicates data missing or illegible when filed

Extended Data Table 6
H3N2 Southern Hemisphere

| | | | | Avg. | Avg. | Avg. | Avg. |
|---|---|---|---|---|---|---|---|
| | WHO | Emergenet | Emergenet | HA WHO | HA Enet | NA WHO | NA Enet |
| Season | Reccomendation | Recommendation 1 | Recommendation 2 | Error | Error | Error | Error |
| 2003 | A/Moscow/10/99 | A/Netherlands/120/2002 | A/Hong_Kong/CUHK24054/ | 24.8 | 7.6 | 13.7 | 3.2 |
| | | | 2002 | | | | |
| 2004 | A/Fujian/411/2002 | A/Wellington/3/2003 | A/Netherlands/88/2003 | 6.4 | 4.4 | 10.0 | 8.9 |
| 2005 | A/Wellington/1/2004 | A/Queensland/40/2003 | A/Hong_Kong/CUHK50372/ | 3.8 | 3.0 | 2.8 | 1.6 |
| | | | 2004 | | | | |
| 2006 | A/California/7/2004 | A/Canterbury/18/2004 | A/Hong_Kong/CUHK7711/ | 13.0 | 2.5 | 4.5 | 1.5 |
| | | | 2005 | | | | |
| 2007 | A/Wisconsin/67/2005 | A/Mexico/DIF2160/2005 | A/New_York/1034/2006 | 8.2 | 2.5 | 7.2 | 6.6 |
| 2008 | A/Brisbane/10/2007 | A/Virginia/UR06-0021/ | A/Madagascar/2694/2006 | 3.7 | 2.6 | 3.2 | 3.2 |
| | | 2007 | | | | | |
| 2009 | A/Brisbane/10/2007 | A/New_York/UR07- | A/Tiajin/Hedong/112/2008 | 5.7 | 4.7 | 3.4 | 1.5 |
| | | 0093/2008 | | | | | |
| 2010 | A/Perth/16/2009 | A/Kentucky/UR07-0148/ | A/Hong_Kong/H090-755/V2 | 5.0 | 5.0 | 2.9 | 2.9 |
| | | 2008 | | | | | |
| 2011 | A/Perth/16/2009 | A/Pennsylvania/24/2009 | A/Texas/90/2009 | 7.0 | 4.2 | 3.4 | 3.4 |
| 2012 | A/Perth/16/2009 | A/Philippines/04/2010 | A/Singapore/N160/2009 | 11.6 | 8.5 | 6.5 | 3.8 |
| 2013 | A/Victoria/361/2011 | A/Peru/PER117/2012 | A/Indiana//20/2012 | 6.9 | 8.1 | 3.5 | 1.4 |
| 2014 | A/Texas/50/2012 | A/Boston/DOAA2-162/2012 | A/Singapore/H2010.586/ | 5.5 | 2.4 | 3.8 | 3.5 |
| | | | 2010 | | | | |
| 2015 | A/Switzerland/9715293/ | A/Nepal1012C/2013 | A/Maryland/03/2014 | 8.9 | 1.5 | 2.6 | 6.6 |
| | 2013 | | | | | | |
| 2016 | A/Hong Kong/4801/2014 | A/Wisconsin/16/2015 | A/Singapore/TT151/2014 | 2.8 | 1.5 | 8.0 | 2.5 |
| 2017 | A/Hong Kong/4801/2014 | A/SAGAMIHARA/1/2016 | A/Vermont/20/2016 | 3.9 | 3.5 | 9.0 | 2.7 |
| 2018 | A/Singapore/INFIMH-16- | A/Darwin/1002/2016 | A/Brazil/0809/2016 | 7.9 | 5.3 | 4.7 | 3.7 |
| | 0019/2016 | | | | | | |
| 2019 | A/Switzerland/8060/2017 | A/Oklahoma/6966/2018 | A/Singapore/EN0480/ | 12.9 | 8.6 | 8.0 | 5.3 |
| | | | 2017 | | | | |
| 2020 | A/South Australia/34/ | A/Jamaica/0488/2019 | A/TOKYO/19063/2019 | 11.5 | 4.8 | 5.3 | 3.5 |
| | 2019 | | | | | | |
| 2021 | A/Hong Kong/2671/2019 | A/Cameroon/8414/2019 | A/Oklahoma/8992/2019 | 15.4 | 11.7 | 4.2 | 3.5 |
| 2022 | A/Darwin/9/2021 | A/Bangladesh/4013/2020 | A/Mali/20027/2020 | 5.5 | 4.2 | 2.4 | 2.0 |
| 2023 | A/Darwin/9/2021 | A/Victoria/3959/2022 | A/Navarra/9534/2021 | −1 | −1 | −1 | −1 |

Avg. error computed as the average minimum edit distance between our two recommendations and the dominant strains, which is defined as the strain closest to the centroid of each cluster in the edit distance metric. The average error is weighted by the sizes of the clusters the dominant strains belong to.

Extended Data Table 7
Out-Performance of Emergenet Recommendations over Randomly Selected Strains

| | | | Two decades | | | One decade | | |
|---|---|---|---|---|---|---|---|---|
| | | | WHO | Enet | Improve- | WHO | Enet | Improve- |
| Subtype | Gene | Hemisphere | Error | Error | ment (%) | Error | Error | ment (%) |
| H1N1 | HA | North | 12.04 | 9.76 | 23.37 | 7.00 | 4.14 | 68.89 |
| H1N1 | HA | South | 12.81 | 8.70 | 47.36 | 6.72 | 4.02 | 67.23 |
| H1N1 | HA | Average | 12.43 | 9.23 | 34.67 | 6.86 | 4.08 | 68.07 |
| H3N2 | HA | North | 8.82 | 5.53 | 59.56 | 10.12 | 6.40 | 58.01 |
| H3N2 | HA | South | 8.45 | 4.83 | 75.14 | 9.30 | 5.16 | 80.27 |
| H3N2 | HA | Average | 8.64 | 5.18 | 66.82 | 9.71 | 5.78 | 67.95 |
| H1N1 | NA | North | 8.79 | 7.64 | 15.08 | 4.71 | 3.64 | 29.26 |
| H1N1 | NA | South | 9.84 | 7.48 | 31.54 | 4.85 | 3.72 | 30.40 |
| H1N1 | NA | Average | 9.31 | 7.56 | 23.23 | 4.78 | 3.68 | 29.84 |
| H3N2 | NA | North | 4.59 | 3.56 | 29.03 | 4.50 | 3.40 | 32.05 |
| H3N2 | NA | South | 5.16 | 3.56 | 44.80 | 4.72 | 3.47 | 36.00 |
| H3N2 | NA | Average | 4.87 | 3.56 | 36.92 | 4.61 | 3.44 | 34.05 |

Extended Data Table 8
Influenza A Strains Evaluated by IRAT and Corresponding Emergenet Computed Risk Scores at Present Time

| | | | IRAT | | | | | Enet | Enet | | |
|---|---|---|---|---|---|---|---|---|---|---|---|
| | | | Emer- | IRAT | | | [symbol] | Emer- | Emer- | Enet | Enet |
| | | IRAT | gence | Impact | HA | NA | Mean | gence | gence | Impact | Impact |
| Influenza Virus | [symbol] | Date | Score | Score | Sample | Sample | E-risk | Score | Err. | Score | Err. |
| A/ [symbol] | H1N1 | July 2020 | 7.5 | [symbol] | [symbol] | [symbol] | [symbol] | [symbol] | [symbol] | [symbol] | [symbol] |
| A/ [symbol] | H3N2 | July 2019 | 5.8 | [symbol] | [symbol] | [symbol] | [symbol] | [symbol] | [symbol] | [symbol] | [symbol] |
| A/Hong Kong [symbol] | H7N9 | May 2017 | 6.5 | 7.5 | 1248 | 1247 | [symbol] | [symbol] | [symbol] | [symbol] | [symbol] |
| A/ [symbol] | H7N9 | April 2016 | [symbol] | 7.2 | [symbol] | [symbol] | [symbol] | [symbol] | [symbol] | [symbol] | [symbol] |
| A/ [symbol] | [symbol] | July 2019 | 5.2 | 5.9 | 58 | 58 | [symbol] | [symbol] | [symbol] | [symbol] | 0.1177 |
| A/ [symbol] | [symbol] | December 2012 | 6.0 | 4.5 | [symbol] | [symbol] | [symbol] | [symbol] | [symbol] | [symbol] | [symbol] |
| A/California [symbol] | H1N2 | July 2019 | 5.8 | 5.7 | 37 | 37 | [symbol] | [symbol] | [symbol] | [symbol] | [symbol] |
| A/ [symbol] | [symbol] | February 2014 | 5.5 | [symbol] | 58 | 58 | [symbol] | [symbol] | [symbol] | [symbol] | [symbol] |
| A/ [symbol] | [symbol] | October 2021 | 5.3 | [symbol] | 45 | 45 | [symbol] | [symbol] | [symbol] | [symbol] | [symbol] |
| A/ [symbol] | [symbol] | November 2011 | 5.2 | [symbol] | [symbol] | 45 | [symbol] | [symbol] | [symbol] | [symbol] | [symbol] |
| A/ [symbol] | [symbol] | April 2015 | 5.0 | [symbol] | 46 | 46 | [symbol] | [symbol] | [symbol] | [symbol] | [symbol] |
| A/ [symbol] | [symbol] | March 2021 | 4.5 | [symbol] | [symbol] | [symbol] | [symbol] | [symbol] | [symbol] | [symbol] | [symbol] |
| A/Netherlands [symbol] | [symbol] | June 2012 | [symbol] | [symbol] | [symbol] | 1247 | 0.2735 | [symbol] | [symbol] | [symbol] | [symbol] |
| A/American/ [symbol] | [symbol] | March 2022 | [symbol] | [symbol] | [symbol] | [symbol] | [symbol] | [symbol] | [symbol] | [symbol] | [symbol] |
| South Carolina [symbol] | | | | | | | | | | | |
| A/ [symbol] | [symbol] | February 2014 | 4.3 | [symbol] | [symbol] | [symbol] | [symbol] | [symbol] | [symbol] | [symbol] | [symbol] |
| A/ [symbol] | [symbol] | March 2015 | 4.2 | [symbol] | [symbol] | [symbol] | [symbol] | [symbol] | [symbol] | [symbol] | [symbol] |
| A/ [symbol] | H5N2 | March 2015 | 3.8 | [symbol] | [symbol] | [symbol] | [symbol] | [symbol] | [symbol] | [symbol] | [symbol] |
| A/ [symbol] | H3N2 | June 2015 | 3.7 | 3.7 | [symbol] | [symbol] | [symbol] | [symbol] | [symbol] | [symbol] | [symbol] |
| A/American [symbol] | H5N1 | March 2015 | [symbol] | 4.1 | [symbol] | 45 | [symbol] | 3.8321 | [symbol] | [symbol] | [symbol] |
| A/ [symbol] | H7N8 | July 2017 | 3.4 | 3.9 | 1248 | 1247 | 0.1350 | [symbol] | [symbol] | 4.9103 | 0.0242 |
| A/ [symbol] | [symbol] | October 2017 | 3.1 | 3.5 | 1248 | 1247 | 0.1314 | [symbol] | 0.2022 | [symbol] | [symbol] |
| A/ [symbol] | [symbol] | October 2017 | 2.8 | 3.5 | 1248 | 1247 | [symbol] | 4.0025 | [symbol] | [symbol] | [symbol] |

[symbol]

indicates data missing or illegible when filed

Extended Data Table 9: Count of Identified Strains
Above Estimated Risk Threshold

| subtype | 6.0 | 6.2 | 6.3 | 6.4 | 6.5 |
|---|---|---|---|---|---|
| H1N1 | 64 | 57 | 54 (77.14%) | 11 (55.0%) | 4 (44.44%) |
| | (76.19%) | (74.03%) | | | |
| H3N2 | 17 | 17 | 15 (21.43%) | 8 (40.0%) | 4 (44.44%) |
| | (20.24%) | (22.08%) | | | |
| H7N9 | 1 (1.19%) | 1 (1.3%) | 1 (1.43%) | 1 (5.0%) | 1 (11.11%) |
| H9N2 | 2 (2.38%) | 2 (2.6%) | 0 (0.0%) | 0 (0.0%) | 0 (0.0%) |

Extended Data Table 10: Influenza A Strains Evaluated by IRAT and Corresponding
Emergenet Computed Risk Scores

| | Sub- | HA | NA | Predicted IRAT | Predicted IRAT |
|---|---|---|---|---|---|
| Strain | type | accession | accession | Impact | Emergence |
| A/swine/Missouri/A02524711/2020 | H1N1 | EPI1818121 | EPI1818122 | 6.7673 | 6.7822 |
| A/swine/Indiana/A02524710/2020 | H3N2 | EPI1818137 | EPI1818138 | 6.7205 | 6.7293 |
| A/swine/North Carolina/02479173/2020 | H1N1 | EPI1780425 | EPI1780426 | 6.7136 | 6.7215 |
| A/Camel/Inner Mongolia/XL/2020 | H7N9 | EPI2026200 | EPI2026202 | 6.6990 | 6.7049 |
| A/swine/Tennessee/A02524414/2022 | H1N1 | EPI2149257 | EPI2149258 | 6.6501 | 6.6494 |
| A/swine/Nebraska/A02245526/2020 | H3N2 | EPI1780195 | EPI1780196 | 6.6058 | 6.5991 |
| A/swine/Minnesota/A02635976/2021 | H1N1 | EPI1912208 | EPI1912209 | 6.5776 | 6.5670 |
| A/swine/Chile/VN1401-5054/2020 | H3N2 | EPI1974975 | EPI1974978 | 6.5318 | 6.5149 |
| A/swine/Italy/56910/2020 | H3N2 | EPI2142217 | EPI2142173 | 6.5292 | 6.5119 |
| A/swine/Minnesota/A02245643/2020 | H3N2 | EPI1769178 | EPI1769179 | 6.5067 | 6.4863 |
| A/swine/Iowa/A02479005/2020 | H1N1 | EPI1777621 | EPI1777622 | 6.4872 | 6.4641 |
| A/swine/Iowa/02524878/2020 | H3N2 | EPI1907866 | EPI1907867 | 6.4566 | 6.4291 |
| A/swine/Indiana/A02636638/2022 | H1N1 | EPI2153370 | EPI2153371 | 6.4534 | 6.4255 |
| A/swine/Iowa/A02524874/2020 | H3N2 | EPI1907838 | EPI1907839 | 6.4392 | 6.4093 |
| A/swine/Minnesota/A02248037/2021 | H1N1 | EPI1912188 | EPI1912189 | 6.4366 | 6.4063 |
| A/swine/Iowa/A02635917/2021 | H1N1 | EPI1911753 | EPI1911754 | 6.4356 | 6.4052 |
| A/swine/Illinois/A02635936/2021 | H1N1 | EPI1911791 | EPI1911792 | 6.4347 | 6.4042 |
| A/swine/Minnesota/A02711801/2022 | H1N1 | EPI2153420 | EPI2153421 | 6.4334 | 6.4027 |
| A/swine/South_Dakokta/A02524453/2020 | H1N1 | EPI1765555 | EPI1765556 | 6.4321 | 6.4012 |
| A/swine/Illinois/A02479007/2020 | H3N2 | EPI1777629 | EPI1777630 | 6.4315 | 6.4005 |
| A/swine/Minnesota/A02248061/2021 | H1N1 | EPI1912494 | EPI1912495 | 6.4278 | 6.3963 |
| A/swine/Iowa/A02524875/2020 | H1N1 | EPI1907858 | EPI1907859 | 6.4260 | 6.3943 |
| A/swine/Spain/44579-1/2020 | H3N2 | EPI1930744 | EPI1930748 | 6.4255 | 6.3937 |
| A/swine/Iowa/A02636439/2022 | H1N1 | EPI2147475 | EPI2147476 | 6.4250 | 6.3931 |
| A/swine/Minnesota/A02248060/2021 | H1N1 | EPI1912500 | EPI1912501 | 6.4234 | 6.3913 |
| A/swine/Nebraska/A02636117/2021 | H1N1 | EPI1932937 | EPI1932938 | 6.4226 | 6.3903 |
| A/swine/Iowa/A02524513/2020 | H1N1 | EPI1832647 | EPI1832648 | 6.4223 | 6.3901 |
| A/swine/Iowa/02524646/2020 | H1N1 | EPI1817164 | EPI1817165 | 6.4222 | 6.3899 |
| A/swine/Nebraska/A02479337/2020 | H1N1 | EPI1769116 | EPI1769117 | 6.4222 | 6.3899 |
| A/swine/Iowa/A02479383/2020 | H1N1 | EPI1771027 | EPI1771028 | 6.4222 | 6.3899 |
| A/swine/Nebraska/A02479212/2020 | H1N1 | EPI1775884 | EPI1775885 | 6.4222 | 6.3899 |
| A/swine/Minnesota/A02479051/2020 | H1N1 | EPI1778572 | EPI1778573 | 6.4222 | 6.3899 |
| A/swine/Minnesota/A02245424/2020 | H1N1 | EPI1780207 | EPI1780208 | 6.4222 | 6.3899 |
| A/swine/Iowa/A02524724/2020 | H1N1 | EPI1818387 | EPI1818388 | 6.4222 | 6.3899 |
| A/swine/Missouri/A02525065/2021 | H1N1 | EPI1908581 | EPI1908582 | 6.4222 | 6.3899 |
| A/swine/Iowa/A02525313/2021 | H1N1 | EPI1910761 | EPI1910762 | 6.4222 | 6.3899 |
| A/swine/Iowa02524994/2020 | H1N1 | EPI1908427 | EPI1908428 | 6.4222 | 6.3899 |
| A/swine/Iowa/A02525345/2021 | H1N1 | EPI1910807 | EPI1910808 | 6.4222 | 6.3899 |
| A/swine/Iowa/A02524892/2020 | H1N1 | EPI1907881 | EPI1907882 | 6.4222 | 6.3899 |
| A/swine/Nebraska.A02524935/2020 | H1N1 | EPI1908118 | EPI1908119 | 6.4222 | 6.3899 |
| A/swine/Missouri/A02524951/2020 | H1N1 | EPI1908429 | EPI1908430 | 6.4222 | 6.3899 |
| A/swine/Nebraska/A02479186/2020 | H1N1 | EPI1774141 | EPI1774142 | 6.4220 | 6.3897 |
| A/swine/Minnesota/A02245699/2020 | H3N2 | EPI1833007 | EPI1833008 | 6.4220 | 6.3897 |
| A/swine/Iowa/A02479156/2020 | H1N1 | EPI1780249 | EPI1780250 | 6.4216 | 6.3892 |
| A/swine/Iowa/A02479229/2020 | H1N1 | EPI1775914 | EPI1775915 | 6.4215 | 6.3891 |
| A/swine/Iowa/A02479303/2020 | H1N1 | EPI1768639 | EPI1768640 | 6.4207 | 6.3882 |
| A/swine/Minnesota/A02471069/2021 | H1N1 | EPI2146090 | EPI2146091 | 6.4200 | 6.3874 |
| A/swine/Iowa/A02635881/2021 | H1N1 | EPI1911668 | EPI1911669 | 6.4198 | 6.3872 |
| A/swine/Iowa/A02525354/2021 | H1N1 | EPI1910789 | EPI1910790 | 6.4198 | 6.3872 |
| A/swine/Iowa/A02635823/2021 | H1N1 | EPI1911263 | EPI1911264 | 6.4176 | 6.3847 |
| A/swine/Iowa/A02524968/2020 | H1N1 | EPI1908419 | EPI1908420 | 6.4173 | 6.3843 |
| A/swine/Iowa/A02479141/2020 | H1N1 | EPI1790241 | EPI1780242 | 6.4161 | 6.3829 |
| A/swine/Nebraska/02525367/2021 | H1N1 | EPI1910817 | EPI1910818 | 6.4136 | 6.3801 |
| A/swine/Iowa/A02245587/2020 | H1N1 | EPI1775817 | EPI1775818 | 6.4119 | 6.3781 |
| A/swine/Iowa/A02750621/2022 | H1N1 | EPI2161576 | EPI2161577 | 6.4116 | 6.3779 |
| A/swine/Iowa/A02636496/2022 | H1N1 | EPI2148086 | EPI2148087 | 6.4105 | 6.3765 |
| A/swine/South Dakota/A02524914/2020 | H3N2 | EPI1908070 | EPI1908071 | 6.4101 | 6.3761 |
| A/swine/Iowa/A02525217/2021 | H1N1 | EPI1909087 | EPI1909088 | 6.4090 | 6.3748 |
| A/swine/Iowa/A02636145/2021 | H1N1 | EPI1932055 | EPI1932930 | 6.4090 | 6.3748 |
| A/swine/Iowa/A02636114/2021 | H1N1 | EPI1931853 | EPI1931854 | 6.4090 | 6.3748 |
| A/swine/Iowa/A02635871/2021 | H1N1 | EPI1911656 | EPI1911657 | 6.4089 | 6.3747 |
| A/swine/Iowa/A02479067/2020 | H1N1 | EPI1778734 | EPI1778735 | 6.4083 | 6.3741 |
| A/swine/Minnesota/A02246459/2021 | H1N1 | EPI1912518 | EPI1912519 | 6.4061 | 6.3716 |
| A/swine/Minnesota/A02711797/2022 | H3N2 | EPI2153382 | EPI2153383 | 6.4029 | 6.3679 |
| A/swine/Minnesota/A02525348/2021 | H1N1 | EPI1910795 | EPI1910796 | 6.3996 | 6.3641 |
| A/swine/Iowa/A02479343/2020 | H1N1 | EPI1769114 | EPI1769115 | 6.3985 | 6.3628 |
| A/swine/Illinois/A02525253/2021 | H3N2 | EPI1910375 | EPI1910376 | 6.3974 | 6.3615 |
| A/swine/Colorado02710706/2022 | H3N2 | EPI2176699 | EPI2176700 | 6.3629 | 6.3221 |
| A/swine/Kansas/A02245381/2020(H1N1) | H1N1 | EPI1777723 | EPI1777724 | 6.3447 | 6.3013 |
| A/canine/Texas/21-011409-001/2021 | H3N2 | EPI1896555 | EPI1896557 | 6.3440 | 6.3005 |
| A/swine/Iowa/A02525161/2021 | H3N2 | EPI1909023 | EPI1909024 | 6.3442 | 6.2893 |
| A/swine/Iowa/A02246996/2021 | H1N1 | EPI2146133 | EPI2146134 | 6.3269 | 6.2809 |
| A/Muscovy_duck/Vietnam/QN6297/2020 | H3N2 | EPI1974815 | EPI1974818 | 6.3239 | 6.2775 |
| A/mink/China/chick_embryo_2020 | H9N2 | EPI2161544 | EPI2161548 | 6.3188 | 6.2716 |

Supplementary Table 1: Number of Influenza Sequences Collected
from Public Databases

| | Influenza | No. HA | No. NA | |
|---|---|---|---|---|
| Database | Subtype | Sequences | Sequences | Total |
| GISAID | H1N1 | 62,230 | 62,242 | 124,472 |
| GISAID | H1N2 | 857 | 857 | 1,714 |
| GISAID | H3N2 | 99,647 | 99,671 | 199,318 |
| GISAID | H5N1 | 1,970 | 1,943 | 3,913 |
| GISAID | H5N2 | 22 | 24 | 46 |
| GISAID | H5N6 | 186 | 186 | 372 |
| GISAID | H5N8 | 1,449 | 1,401 | 2,850 |
| GISAID | H7N1 | 3 | 3 | 6 |
| GISAID | H7N2 | 2 | 2 | 4 |
| GISAID | H7N3 | 101 | 99 | 200 |
| GISAID | H7N5 | 1 | 1 | 2 |
| GISAID | H7N6 | 8 | 8 | 16 |
| GISAID | H7N7 | 57 | 56 | 113 |
| GISAID | H7N8 | 4 | 4 | 8 |
| GISAID | H7N9 | 1,265 | 1,264 | 2,529 |
| GISAID | H9N2 | 312 | 312 | 624 |
| GISAID | H10N8 | 1 | 1 | 2 |
| NCBI | H1N1 | 18,577 | 16,913 | 35,490 |
| NCBI | H3N2 | 18,840 | 15,249 | 34,089 |
| NCBI | H5N1 | 1 | 1 | 2 |
| NCBI | H5N8 | 1 | 1 | 2 |
| NCBI | H7N7 | 1 | 1 | 2 |
| NCBI | H7N9 | 2 | 2 | 4 |
| Both | All | 205,537 | 200,241 | 405,778 |

Supplementary Table 2
Examples: Emergenet Induced Distance Varying for Fixed Sequence Pair when Background
Population Changes (Rows 1-5), Sequences with Small Edit Distance and Large E-distance,
and the Converse (Rows 6-9)

| | Edit | | | Emergenet | | |
|---|---|---|---|---|---|---|
| | dist. | Sequence A | Sequence B | E-dist. | Year A* | Year B* |
| 1 | 18 | A/Singapore/23J/2007 | A/Tennessee/UR06-0294/2007 | 0.0111 | 2007 | 2007 |
| 2 | 18 | A/Singapore/23J/2007 | A/Tennessee/UR06-0294/2007 | 0.0094 | 2008 | 2008 |
| 3 | 18 | A/Singapore/23J/2007 | A/Tennessee/UR06-0294/2007 | 0.0027 | 2009 | 2009 |
| 4 | 18 | A/Singapore/23J/2007 | A/Tennessee/UR06-0294/2007 | 0.0025 | 2010 | 2010 |
| 5 | 18 | A/Singapore/23J/2007 | A/Tennessee/UR06-0294/2007 | 0.6163 | 2007 | 2010 |
| 6 | 11 | A/Naypyitaw/M783/2008 | A/Singapore/201/2008 | 0.8852 | 2008 | 2008 |
| 7 | 15 | A/Cambodia/W0908339/2012 | A/Singapore/DMS1233/2012 | 0.2737 | 2012 | 2012 |
| 8 | 126 | A/South Dakota/03/2008 | A/Singapore/10/2008 | 0.3034 | 2008 | 2008 |
| 9 | 141 | A/Jodhpur/3248/2012 | A/Cambodia/W0908339/2012 | 0.2405 | 2012 | 2012 |

*Year A and year B correspond to the assumed collector years for sequences A and B respectively for the purpose of this example. Sequence A in row 1 is collected in 2007, but is assumed to be from different years in rows 2-4 to demonstrate the change in E-distance from sequence B, arising only from a change in the background population.

Supplementary Table 3: Correlation between E-distance
and Edit Distance Between Sequence Pairs

| Phenotypes | Correlation |
|---|---|
| Influenza H1N1 HA | 0.76 |
| Influenza H1N1 NA | 0.74 |
| Influenza H3N2 HA | 0.85 |
| Influenza H3N2 NA | 0.79 |

Supplementary Table 4
General Linear Model Evaluating Emergenet Emergence
Risk Predictions Against IRAT Estimates
Mode1:

[symbol]

| | | | |
|---|---|---|---|
| [symbol] Variable: | IRAT_ Emergence_Score | No. [symbol] | [symbol] |
| [symbol] | [symbol] | [symbol] | [symbol] |
| [symbol] | [symbol] | [symbol] | [symbol] |
| Link Function: | Identity | Scale: | [symbol] |
| Method: | [symbol] | Log-Likelihood: | [symbol] |
| Date: | Tue, 14 Mar. 2023 | Deviance: | [symbol] |
| Time: | [symbol] | [symbol] | [symbol] |
| No. [symbol] | 3 | [symbol] | [symbol] |
| [symbol] Type: | nonrobust | | |

| | [symbol] | [symbol] | [symbol] | [symbol] | [symbol] | [symbol] |
|---|---|---|---|---|---|---|
| Intercept | [symbol] | [symbol] | [symbol] | [symbol] | [symbol] | [symbol] |
| Geometric_Mean | [symbol] | [symbol] | [symbol] | [symbol] | [symbol] | [symbol] |

Mode1:

[symbol]

| | | | |
|---|---|---|---|
| [symbol] Variable: | IRAT_ Emergence_Score | No. [symbol] | [symbol] |
| [symbol] | [symbol] | [symbol] | [symbol] |
| [symbol] | [symbol] | [symbol] | [symbol] |
| Link Function: | Identity | Scale: | [symbol] |
| Method: | [symbol] | Log-Likelihood: | [symbol] |
| Date: | Tue, 14 Mar. 2023 | Deviance: | [symbol] |
| Time: | [symbol] | [symbol] | [symbol] |
| No. [symbol] | 3 | [symbol] | [symbol] |
| [symbol] Type: | nonrobust | | |

| | [symbol] | [symbol] | [symbol] | [symbol] | [symbol] | [symbol] |
|---|---|---|---|---|---|---|
| Intercept | [symbol] | [symbol] | [symbol] | [symbol] | [symbol] | [symbol] |
| Geometric_Mean | [symbol] | [symbol] | [symbol] | [symbol] | [symbol] | [symbol] |
| [symbol] | [symbol] | [symbol] | [symbol] | [symbol] | [symbol] | [symbol] |

[symbol]

indicates data missing or illegible when filed

Supplementary Table 5
General Linea Model Evaluating Emergenet Impact
Risk Predictions Against IRAT Estimates
Mode1:

[symbol]

| | | | |
|---|---|---|---|
| [symbol] Variable: | [symbol] | No. [symbol] | [symbol] |
| [symbol] | [symbol] | [symbol] | [symbol] |
| [symbol] | [symbol] | [symbol] | [symbol] |
| Link Function: | Identity | Scale: | [symbol] |
| Method: | [symbol] | Log-Likelihood: | [symbol] |
| Date: | Tue, 14 Mar. 2023 | Deviance: | [symbol] |
| Time: | [symbol] | [symbol] | [symbol] |
| No. [symbol] | 3 | [symbol] | [symbol] |
| [symbol] Type: | nonrobust | | |

| | [symbol] | [symbol] | [symbol] | [symbol] | [symbol] | [symbol] |
|---|---|---|---|---|---|---|
| Intercept | [symbol] | [symbol] | [symbol] | [symbol] | [symbol] | [symbol] |
| Geometric_Mean | [symbol] | [symbol] | [symbol] | [symbol] | [symbol] | [symbol] |

Mode1:

[symbol]

| | | | |
|---|---|---|---|
| [symbol] Variable: | [symbol] | No. [symbol] | [symbol] |
| [symbol] | [symbol] | [symbol] | [symbol] |
| [symbol] | [symbol] | [symbol] | [symbol] |
| Link Function: | Identity | Scale: | [symbol] |
| Method: | [symbol] | Log-Likelihood: | [symbol] |
| Date: | Tue, 14 Mar. 2023 | Deviance: | [symbol] |
| Time: | [symbol] | [symbol] | [symbol] |
| No. [symbol] | 3 | [symbol] | [symbol] |
| [symbol] Type: | nonrobust | | |

| | [symbol] | [symbol] | [symbol] | [symbol] | [symbol] | [symbol] |
|---|---|---|---|---|---|---|
| Intercept | [symbol] | [symbol] | [symbol] | [symbol] | [symbol] | [symbol] |
| Geometric_Mean | [symbol] | [symbol] | [symbol] | [symbol] | [symbol] | [symbol] |
| [symbol] | [symbol] | [symbol] | [symbol] | [symbol] | [symbol] | [symbol] |

[symbol]

indicates data missing or illegible when filed

Supplementary Table 6
General Linear Model for Evaluating Effect of
Data Diversity on Emergenet Performance

| [symbol] Name | Description |
|---|---|
| [symbol] | [symbol] number of [symbol] |
| | corresponding Emergents |
| data_diversity | Number of clusters in [symbol] input |
| | [symbol] |
| [symbol] | [symbol] WHO [symbol] |

Mode1:

[symbol]

| | | | |
|---|---|---|---|
| [symbol] Variable: | [symbol] | No. [symbol] | [symbol] |
| [symbol] | [symbol] | [symbol] | [symbol] |
| [symbol] | [symbol] | [symbol] | [symbol] |
| Link Function: | Identity | Scale: | [symbol] |
| Method: | [symbol] | Log-Likelihood: | [symbol] |
| Date: | Tue, 11 Jun. 2022 | [symbol] | [symbol] |
| Time: | [symbol] | [symbol] | [symbol] |
| No. [symbol] | [symbol] | [symbol] Type | [symbol] |

| | [symbol] | [symbol] | [symbol] | [symbol] | [symbol] | [symbol] |
|---|---|---|---|---|---|---|
| Intercept | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

Mode1:

[symbol]

| | | | |
|---|---|---|---|
| [symbol] Variable: | [symbol] | No. [symbol] | [symbol] |
| [symbol] | [symbol] | [symbol] | [symbol] |
| [symbol] | [symbol] | [symbol] | [symbol] |
| Link Function: | Identity | Scale: | [symbol] |
| Method: | [symbol] | Log-Likelihood: | [symbol] |
| Date: | Tue, 14 Mar. 2023 | Deviance: | [symbol] |
| Time: | [symbol] | [symbol] | [symbol] |
| No. [symbol] | 3 | [symbol] | [symbol] |

| | [symbol] | [symbol] | [symbol] | [symbol] | [symbol] | [symbol] |
|---|---|---|---|---|---|---|
| Intercept | [symbol] | [symbol] | [symbol] | [symbol] | [symbol] | [symbol] |
| Geometric_Mean | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

[symbol]

indicates data missing or illegible when filed

Supplementary Table 7: IRAT Predictions that Broadly Corroborated with Outbreaks

| | | IRAT | IRAT | |
|---|---|---|---|---|
| | | Emergence | Impact | |
| Influenza Virus | Subtype | Score | Score | Description |
| A/ [symbol] 37/2016 | H1N1 | 7. [symbol] | [symbol] | Cases of [symbol] saw a substantial increase in |
| | | | | weekly cases in the Shandong providence of China during the years |
| | | | | 2016-2017 when compared to previous years [symbol] . This strain was also |
| | | | | ch [symbol] n as a representative strain in a study by Su [symbol] et al. which |
| | | | | found that its potential for human infectivity “greatly enhances the |
| | | | | opportunity for virus adaptation in humans and raises concerns for |
| | | | | the possible generation of pandemic viruses [symbol] . |
| A/C [symbol] /2017 | H [symbol] N2 | 6. [symbol] | 5. [symbol] | The CDC reports that flu activity in the United States during the |
| | | | | 2017-2018 [symbol] started to increase in November and was |
| | | | | dominated by influenza A (H3N2) viruses through February [symbol] . |
| A/Hong Kong/ | H [symbol] N3 | 6. [symbol] | 7. [symbol] | The CDC reports that “during Mar. 31, 201 [symbol] -Aug. 7, 2017, a |
| 12 [symbol] /2017 | | | | total of 1, [symbol] 7 human infections with Asian H7N [symbol] viruses were |
| | | | | reported [symbol] at least [symbol] of these infections [symbol] in death.” |
| | | | | 7 [symbol] of these infections were reported during the fifth epidemic |
| | | | | (Oct. 1, 201 [symbol] -Aug. 7, 2017 [symbol] . |

[symbol]

indicates data missing or illegible when filed

Supplementary Table 8: Sampling Effects on H1N1 HA Predictions, 2010-2023

| Season | Avg. HA WHO Error | Avg. HA E [symbol] Error | E [symbol] Improvement | Std. HA E [symbol] Error |
|---|---|---|---|---|
| 2010-11 | 5.515 | 2.414 | 3.100 | 0. [symbol] 39 |
| 2011-12 | 7.505 | 3.40 [symbol] | 4.097 | 0.2 [symbol] 0 |
| 2012-13 | 11.293 | 4. [symbol] 78 | 6.716 | 0. [symbol] 79 |
| 2013-14 | 11.821 | 2. [symbol] 11 | 9.010 | 0.760 |
| 2014-15 | 16.110 | 5. [symbol] 97 | 10.714 | 0.4 [symbol] |
| 2015-16 | 16.337 | 4. [symbol] 40 | 10.497 | 0.4 [symbol] |
| 2016-17 | 16.190 | 1.7 [symbol] | 14.43 [symbol] | 0.022 |
| 2017-18 | 4.40 [symbol] | 3. [symbol] 99 | 0.607 | 0. [symbol] 99 |
| 2018-19 | [symbol] .155 | 2.155 | 4.000 | 0.6 [symbol] 2 |
| 2019-20 | [symbol] .397 | 4. [symbol] | 3.799 | 0. [symbol] 4 |
| 2020-21 | 5.093 | 5.695 | −0. [symbol] 2 | 0.181 |
| 2021-22 | [symbol] 67 | 5.010 | 4. [symbol] 7 | 1. [symbol] 46 |
| 2022-23 | 10. [symbol] 32 | 4.322 | 6.200 | 0.000 |

The effects of sampling on H1N1 predictions for relevant years are shown (prior to 2010-11, there werew <3000 strains available so no sampling was done) by performing 15 prediction iterations for each season. The sample standard deviation error is much smaller than the Emergenet improvement on the WHO prediction each year except 2020-21, where Emergenet improvement was negative. [symbol]
indicates data missing or illegible when filed

What is claimed:

1. A method comprising:
reading a genetic sequence of a first strain of a virus;
identifying a plurality of residue indices in the genetic sequence;
for each of the plurality of indices, assigning a predictor, the predictor configured to predict a residue for its assigned index based upon a residue of at least one other index, the predictors thereby forming a network;
determining, based on the network of predictors, a probability of transition of the first strain to a second strain.

2. The method of claim 1, further comprising:
determining, based on the probability of transition, that the second strain is above a dominant strain threshold; and
providing a forecast of the dominant strain for a subsequent season.

3. The method of claim 2, wherein the dominant strain is a strain with a maximized probability of simultaneously arising from a set of currently circulating strains.

4. The method of claim 1, wherein the first strain is an animal strain of the virus and the second strain is a human strain of the virus.

5. The method of claim 4, further comprising:
determining a pandemic potential of the first strain based upon the probability of transition of the first strain to a second strain.

6. The method of claim 1, further comprising:
determining a divergence of each residue index of the plurality of residue indices;
averaging the divergence over the plurality of residue indices in the genetic sequence;
determining, based on the average of the divergence, a distance metric corresponding to the genetic sequence.

7. The method of claim 1, further comprising:
based on the probability of transition, determining a risk score of the second strain,
wherein the risk score indicates a risk of transmission to a human.

8. The method of claim 7, wherein the risk score is an average log likelihood of the probability of transition.

9. The method of claim 7, further comprising:
reading a library of strains of the virus;
determining a distance value between the second strain and each of the library of strains; and
determining, from the distance values, a high-risk subset of the library.

10. The method of claim 1, wherein the virus is an influenza.

11. The method of claim 1, wherein the genetic sequence is a genomic sequence.

12. The method of claim 1, wherein each of the residue indices corresponds to one or more surface proteins.

13. The method of claim 1, wherein each predictor comprises a conditional inference tree, a regression tree, and/or a decision tree.

14. The method of claim 1, wherein determining the probability of transition comprises:
inferring a variation of a mutational probability of the sequence and potential residue replacements between the plurality of residue indices; and
determining a probability of a spontaneous transition based on the inferred variation and the potential residue replacement.

15. The method of claim 1, wherein the probability of transition is a conditional distribution.

16. The method of claim 1, wherein the second strain comprises a human strain of a same subtype of the first strain, wherein the first strain is associated with a first season and the second strain is associated with a second season subsequent to the first season.

17. The method of claim 16, further comprising searching a plurality of seasons prior to the first season for one or more historical strains of the same subtype.

18. The method of claim 17, further comprising recomputing the probability of transition for each season.

19. A system comprising:
a datastore;
a computing node comprising a computer readable storage medium having program instructions embodied therewith, the program instructions executable by a processor of the computing node to cause the processor to perform a method comprising:
reading a genetic sequence of a first strain of a virus from the datastore;
identifying a plurality of residue indices in the genetic sequence;
for each of the plurality of indices, assigning a predictor, the predictor configured to predict a residue for its assigned index based upon a residue of at least one other index, the predictors thereby forming a network;
determining, based on the network of predictors, a probability of transition of the first strain to a second strain.

20. A computer program product for determining a probability of transition of a virus, the computer program product comprising a computer readable storage medium having program instructions embodied therewith, the program instructions executable by a processor to cause the processor to perform a method comprising:
reading a genetic sequence of a first strain of a virus;
identifying a plurality of residue indices in the genetic sequence;
for each of the plurality of indices, assigning a predictor, the predictor configured to predict a residue for its assigned index based upon a residue of at least one other index, the predictors thereby forming a network;
determining, based on the network of predictors, a probability of transition of the first strain to a second strain.


SEQUENCE LISTING (14 sequences)

SEQ ID NO: 1 - protein, 25 aa, Influenza A virus
GTSRYSKKFKPEIATRPKVRDQEGR

SEQ ID NO: 2 - protein, 25 aa, Influenza A virus
GTSKYGKKFMPEIARRPKVRNQEGR

SEQ ID NO: 3 - protein, 25 aa, Influenza A virus
GSSKYYKRFTPEIVARPKVREQAGR

SEQ ID NO: 4 - protein, 250 aa, Influenza A virus
GVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNI

SEQ ID NO: 5 - protein, 250 aa, Influenza A virus
GVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNV

SEQ ID NO: 6 - protein, 250 aa, Influenza A virus
GVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNV

SEQ ID NO: 7 - protein, 550 aa, Influenza A virus
MKTIIALSNILCLVFAQKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPGYASLRSLVASSGTLEFKNESFNWTGVKQNGTSSACIRGSSSSFFSRLNWLTSINNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRKGKSSIMRSDAPIGRCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGF

SEQ ID NO: 8 - protein, 550 aa, Influenza A virus
MKTIIALSNILCLVFAQKIPGNDNSTATLCLGHHAVPNGTIVKTITNDRIEVTNATELVQNSSIGEICNSPHQILDGGNCTLIDALLGDPQCDGFQNKEWDLFVERSRANSSCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQFSLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGF

SEQ ID NO: 9 - protein, 550 aa, Influenza A virus
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGF

SEQ ID NO: 10 - protein, 550 aa, Influenza A virus
MKTIIALSYILCLVFAQKIPGNDNGTATLCLGHHAVPNGTIVKTITDDRIEVTNATELVQNSSIGEICDSPHQVLDGGNCTLIDALLGDPQCDGFQNKKWDLFVERSRAYSNCYPYDVPDYASLRSLVASSGTLEFKNESFNWTGVTQNGKSSSCIRGSSSSFFSRLNWLTXLNYTYPALNVIMPNKEQFDKLYIWGVHHPGTDNDQIFLYARSSGRIIVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYIKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGF

SEQ ID NO: 11 - protein, 550 aa, Influenza A virus
MKAILVVLLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAI

SEQ ID NO: 12 - protein, 550 aa, Influenza A virus
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTITDQESLYQNADAYVFVGTSRYSKKFKPEIAARPKVRDQAGRMNYYWTLVEPGDKITFEATGNLVAPRYAFTMEKDAGSGIIISDTPVHDCNTTCQTPEGAINTSLPFQNVHPITIGKCPKYVRSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAI

SEQ ID NO: 13 - protein, 550 aa, Influenza A virus
MKAVLVVLLYTVTNANADTLCIGYHANNSTDTVDTVLEKNVTVTHSVNLLETRHNGKLCKLKGVAPLHLGKCNIAGWLLGNPECESLSTASSWSYIVETSNSENGTCYPGDFINYEELREQLSSVSSFERFEIFPKTSSWPNHETDKGVTAACPHAGTRSFYRNLVWLVQKGNSYPKVNTSYINNKGKEVLVLWAIHHPSTSSDQQSLYKNEDAYVFVGSSRYSRKFEPEIATRPKVRDQAGRMNYYWTLVEPGDKITFEATGNLVAPRYAFALKRNSGSGIIISDASVHDCDTTCQTPNGAINTSLPFQNIHPVTIGECPKYVKSTKLRMATGLRNIPSIQSRGLFGAIAGFIEGGWTGMIDGWYGYHHQNEQGSGYAADLKSTQSAIDGITNKVNSVIEKMNTQFTAVGKEFSYLEKRIENLNKKVDDGFLDIWTYNAELLVLLENERTLDYHDSNVKNLYEKVRSQLKNNAKEIGNGCFEFYHKCDDMCMESVKNGTYDYPKYSEEAKLNREEIDGVKLESTRIYQILAIYSTVASSLVLVVSLGAI

SEQ ID NO: 14 - protein, 550 aa, Influenza A virus
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAI